(19) United States
(12) Patent Application Publication
(10) Pub. No.: US 2024/0352110 A1
(43) Pub. Date: Oct. 24, 2024

(54) A NOVEL ANTIBODY FOR DETECTION OF AMYLOID BETA 42 (AB42)

(71) Applicant: Roche Diagnostics Operations, Inc., Indianapolis, IN (US)

(72) Inventors: Alexander Jethwa, München (DE); Tobias Oelschlaegel, Geretsried-Gelting (DE); Corinna Schindler, Wolfratshausen (DE); Monika Soukupova, Wessobrunn (DE); Laura Stoeckl, Penzberg (DE); Christina Zimmermann, Huglfing (DE)

(21) Appl. No.: 18/745,539

(22) Filed: Jun. 17, 2024

Related U.S. Application Data
(63) Continuation of application No. PCT/EP2022/086115, filed on Dec. 15, 2022.

(30) Foreign Application Priority Data
Dec. 17, 2021 (EP) 21215740.8

(51) Int. Cl. C07K 16/18; G01N 33/543; G01N 33/68

(52) U.S. Cl. CPC C07K 16/18; G01N 33/543; G01N 33/6896; C07K 2317/565; G01N 2333/4709; G01N 2470/04

(57) ABSTRACT

The present invention relates to a monoclonal antibody or an antigen-binding fragment thereof specifically binding to Aβ42 with advantageous features for Aβ42 detection in vitro using immunoassays. Also provided is a polynucleotide or a set of polynucleotides encoding the same and a vector comprising said polynucleotide(s). Further provided is a host cell comprising the polynucleotide(s) and a corresponding production process using this host cell. Also provided herein are uses and methods employing the monoclonal antibody or an antigen-binding fragment thereof specifically binding to Aβ42 as provided herein.

The present invention relates to a novel Aβ42-specific antibody, which shows advantageous features in in vitro Aβ42 detection assays and aspects related to said novel Aβ42 specific antibody.

Accumulation of the protein amyloid beta (Aβ), especially the aggregation-prone small peptide Aβ42, in so-called plaques in the brain is a major hallmark of Alzheimer's disease (AD). That is why early detection and quantification of Aβ levels is crucial for diagnosis and potential future treatment of AD. Currently, the two major methods for Aβ detection are (1) PET scan based on radiolabeled tracers that bind to Aβ deposits in the brain, and (2) measurement of the Aβ level in cerebrospinal fluid (CSF), e.g. with Roche Elecsys® tests. Both methods have the disadvantage that they are to a certain extent invasive and not easily available for patients around the world. Due to technology advancements in the recent years, it is now possible to quantify Aβ42 levels also in blood plasma using e.g. mass spectrometry or ultrasensitive immunoassays (Hampel et al., Zetterberg et al., Nat Rev Neurol, 2018. 14(11): p. 639-652). However, there is still a need to further increase the robustness of Aβ42 quantification in blood and to identify assay setups, which further improve clinical performance.

One aspect that can potentially influence robustness of blood based assays are interferences. It is well known that blood contains endogenous substances that may interfere with the components (e.g. antibodies) used in assays for quantifying Aβ42, e.g. heterophilic antibodies directed against the assay components, such as antibodies (Zetterberg, H. and S. C. Burnham, Blood-based molecular biomarkers for Alzheimer's disease. Mol Brain, 2019. 12(1): Art.nr. 26). These types of antibodies are much less of a problem in CSF. For example, known sources for potential interferences in blood based antibody based immunoassays are human rheumatoid factor (Rf) (Tate et al. Clin Biochem Rev. 2004; 25(2):105-120; Ward et al. Clin Biochem. 2017; 50(18):1306-1311), i.e. autoantibodies, most often of the immunoglobulin M (IgM) subtype (M Gioud-Paquet et al., Ann Rheum Dis. 1987 January; 46(1): 65-71), that are commonly found in elevated concentrations in blood from patients suffering from rheumatoid arthritis and other diseases such as lupus or sepsis (Francesca Ingegnoli et al., Dis Markers. 2013; 35(6): 727-734). This may cause false-positive or false-negative assay results and thereby lower the clinical value of such assays (Tate et al.; supra, Ward et al., supra).

Accordingly, there is also a need to improve the robustness of antibody-based assays for detecting Aβ42 in blood samples, e.g. by providing improved antibodies. In particular, it is desirable for detection of Aβ42 in blood samples to minimize interferences such as Rf-induced interferences and/or to increase the clinical performance of such Aβ42 assays alone or in combination with other biomarkers (e.g. an Aβ42/Aβ40 ratio).

The present invention provides a solution to the above-mentioned needs.

According to a first aspect, provided herein is a monoclonal antibody or an antigen-binding fragment thereof specifically binding to Aβ42.

“Specifically binding to Aβ42” as used herein means that the monoclonal antibody or antigen fragment binds to Aβ42 in an antigen-antibody reaction and discriminates Aβ42 from other proteins, in particular, also from other Aβ peptides (e.g. Aβ38, Aβ40 and/or Aβ43) that do not have a C-terminus formed by amino acid 42 of Aβ42. “Discriminates” means that the binding affinity is higher to Aβ42 than to the other proteins or peptides.

The specificity of the antibody or antigen binding-fragment thereof is achieved by the fact that the C-terminus of Aβ42 is comprised in the epitope recognized by the antibody. Accordingly, the antibody or antigen-binding fragment of the invention may bind to the C-terminus of Aβ42.

In embodiments, “specifically binding to Aβ42” may mean that the antibody or antigen-binding fragment of the invention bind Aβ42 with an affinity that is at least 5-fold, preferably 10 fold, even more preferably 50 fold or most preferably 100 fold higher than for other proteins or peptides (e.g. Aβ38, Aβ40 and/or Aβ43). In other words, the dissociation equilibrium constant K_{D} for the binding to Aβ42 is 5-fold, preferably 10 fold, even more preferably 50 fold or most preferably 100 fold lower than for other proteins or peptides (e.g. Aβ38, Aβ40 and/or Aβ43).

In embodiments, the antibody or antigen-binding fragment of the invention discriminates Aβ42 from (all) other Aβ peptides (e.g. Aβ38, Aβ40 and Aβ43) in that the binding affinity for Aβ42 is at least 5-fold, preferably 10 fold, even more preferably 50 fold or most preferably 100 fold higher than for (all) other Aβ peptides (e.g. Aβ38, Aβ40 and Aβ43). In other words, the dissociation equilibrium constant K_{D} for the binding to Aβ42 is 5-fold, preferably 10 fold, even more preferably 50 fold or most preferably 100 fold lower than for (all) other Aβ peptides (e.g. Aβ38, Aβ40 and Aβ43).

The binding affinity and/or dissociation equilibrium constant K_{D} for the binding to Aβ42 may be determined by methods known in the art such as surface plasmon resonance spectroscopy (e.g. using the settings as described herein below and/or in the appended Examples).

In embodiments, an antibody may be considered to specifically bind to Aβ42, when measuring a predetermined concentration of Aβ42 in a sample is not affected by spiking in Aβ38, Aβ40 and/or Aβ43 at an excess concentration of 2 fold, preferably 10 fold and even more preferably 50 fold compared to the predetermined Aβ42 concentration. “Not affected” means that the predetermined concentration is measured correctly considering the measuring error of the respective method (e.g. 5% or 10%).

In embodiments, the antibody or antigen-binding fragment of the invention is further characterized in that an 2 fold, preferably 10 fold and even more preferably 50 fold excess of either Aβ38, Aβ40 and Aβ43 does not compete for the binding to Aβ42.

To determine the competition of Aβ38, Aβ40 and/or Aβ43 with Aβ42 for binding to an antibody or antigen-binding fragment an immunoassay for quantifying Aβ42 (e.g. as described herein elsewhere and in the appended Examples) using competition with Aβ 38, Aβ40 and/or Aβ43 peptides or mimicking peptides thereof may be employed. In particular, an Elecsys® based Aβ42 immunoassay may be used. A non-limiting but preferred Example of such assays is disclosed in the appended Examples.

For determining affinity (or other kinetic parameters) or for competition experiments as described herein Aβ42, Aβ38, Aβ40 and/or Aβ43 mimicking peptides may be used. Such mimicking peptides comprising only parts of the peptides (including the C-terminus as free C-terminus), as it has been found that such shortened mimicking peptides are better soluble. Exemplary mimicking peptides are described in the appended Examples. For Aβ38 a mimicking peptide may comprise amino acids 1-12 and 25-38 of Aβ38 but not amino acids 13 to 24 of Aβ38 (e.g. amino acids 1-12 and 25-38 of Aβ38 connected with a linker). For Aβ40 a mimicking peptide may comprise amino acids 1-12 and 25-40 of Aβ40 but not amino acids 13 to 24 of Aβ40 (e.g. amino acids 1-12 and 25-40 of Aβ40 connected with a linker). For Aβ43 a mimicking peptide may comprise amino acids 1-12 and 34-43 of Aβ43 but not amino acids 13 to 33 of Aβ43 (e.g. amino acids 1-12 and 34-43 of Aβ43 connected with a linker). For all mimicking peptides the C-terminus corresponds to the respective Aβ peptide. The mimicking peptides described are part of the present invention. Aβ42 mimicking peptides are described herein below. In mimicking peptides different linkers in the art may be used. A non-limiting example is the following linker: -O2Oc-O2Oc-Lys(Bi)-O2Oc-O2Oc-.

The monoclonal antibody or antigen-binding fragment of the invention has been found to surprisingly outperform previously used monoclonal Aβ42-specific antibodies when used in an immunoassay for detecting Aβ42 in blood samples, especially when used in combination with an Aβ42/Aβ40 ratio. Specifically, an immunoassay using the novel Aβ42 specific antibody of the invention showed a significantly better clinical performance in assessing amyloid positivity when used in an Aβ42/Aβ40 ratio based on blood samples than Aβ42 antibodies that are widely used in prior art assays in a similar assay formats.

Accordingly, in embodiments, the monoclonal antibody or an antigen-binding fragment thereof specifically binding to Aβ42 of the invention is characterized in that when it is used in a sandwich immunoassay for detecting and/or quantifying Aβ42 in blood samples (e.g. serum or plasma samples) the accuracy for determining amyloid positivity in samples of a reference population using an Aβ42/Aβ40 ratio (or vice versa) is higher than for the same Aβ42 sandwich immunoassay using prior art Aβ42 antibodies 21F12 and/or H31L21. The accuracy is preferably determined by a ROC analysis using the AUC (area under the curve) for detecting amyloid positivity in the samples of the reference population (with known amyloid status) as measure for accuracy. In embodiments, the AUC of the Aβ42/Aβ40 ratio for detecting amyloid positivity in the samples of the reference population may be at least 0.8, preferably at least 0.85 and most preferably at least 0.86.

In embodiments, the monoclonal antibody or an antigen-binding fragment of the invention, wherein when used in a sandwich immunoassay for Aβ42 in blood samples (e.g. serum or plasma samples) the accuracy for determining amyloid positivity in samples of a reference population with predetermined amyloid status using an Aβ42/Aβ40 ratio (or vice versa) is at least as good as for the same sandwich immunoassay using the antibody of the invention having a heavy chain of SEQ ID NO:9 and a light chain of SEQ ID NO:10. The accuracy is preferably determined by a ROC analysis and the AUC (area under the curve) for detecting amyloid positivity in the samples of the reference population (with known amyloid status) is used as measure for accuracy.

The reference population may, for example be a reference cohort as used in the appended Examples, e.g. may comprise amyloid positive and amyloid negative subjects. The amyloid positive subjects may have no, mild cognitive impairment or cognitive impairment.

The immunoassay as used in any of the Rf interference analysis may be a sandwich immunoassay, e.g. an Elecsys® immunoassay as described in the appended examples.

The immunoassay may use an Aβ-specific binding agent specifically binding to any of amino acids 1 to 42 (i.e. Aβ specific but not Aβ42 specific) and not competing with binding to Aβ42 with the antibody or antigen-binding fragment of the invention as a second binding agent. Exemplary but not limiting Examples for such second binding agents are 3D6, 6E10 (BioLegend cat. no. 803004) and 1E8 (Nanotools cat. no. 0315-100/bA4N-1E8). In a preferred embodiment, the second binding agent is 3D6, as defined herein below.

The reference read-out for amyloid positivity used for the ROC analysis may be (e.g. Elecsys® CSF pTaul81/Aβ42 ratios as measured in CSF samples, for example using a cut-off of 0.024) or PET scan analysis. In preferred embodiments, the reference read-out for amyloid positivity used for the ROC analysis is Elecsys® CSF pTaul81/Aβ42 ratios as measured in CSF samples, preferably using a cut-off of 0.024.

As one factor contributing to the improved clinical outcome for blood based Aβ42 immunoassays using the antibody of the invention, the present inventors have surprisingly found that the antibody of the invention is robust with respect to rheumatoid factor (Rf) interference in blood samples compared to the most Aβ42 antibodies presently widely used in the field: 21F12 and H31L21 (Pannee J et al., Alzheimers Dement (Amst) 2021 Oct. 14; 13(1):e12242. doi: 10.1002/dad2.12242). During characterization of the features of the antibody of the present invention and comparison with the mentioned prior art antibodies, a previously undescribed interference with Rf has been identified for 21F12 and H31L21 but not the antibody of the invention. The interference was initially identified in samples artificially spiked with a purified Rf from a pool of blood plasmas as well as in natural samples with elevated Rf levels. Contrary to conventional Rf interference, the interference does not depend on the Fc domain of the respective antibodies but could be allocated to an antigen-binding fragment (e.g. an F(ab)₂ fragment) comprising the variable domains (VH and VL) of these prior art antibodies. There is ample evidence that the VH and VL domains of the prior art antibodies, and more specifically the CDR residues thereof contribute to the Rf interference (see Examples and Figures). The inventors could further demonstrate that Rf IgM antibodies are bound to the prior art antibody 21F12 when using samples with elevated IgM antibodies, said binding to a large extent correlating with the observed Rf interference in immunoassays using 21F12. This finding suggest that prior art Aβ42 antibodies suffer from an Rf IgM interference caused by an antigen-binding domain, with a critical role of the VH and VL, more specifically the CDRs comprised therein.

Surprisingly, the novel monoclonal Aβ42 specific antibody (clone 3.2.52) identified herein did not show the Rf interference as observed for the widely used prior art Aβ42 antibodies 21F12 and H31L21. This feature is one factor contributing to the improved clinical performance of the immunoassay using the novel monoclonal antibody of the invention. By overcoming interference with Rf interference in an antibody-based Aβ42 detection method, such as an immunoassay (e.g. an heterogeneous immunoassay), the robustness of Aβ42 detection especially in blood samples, such as plasma, can be increased. As demonstrated in the appended examples, the novel monoclonal antibody provided herein could accurately detect Aβ42 in samples independent of the presence of a purified Rf from a pool of blood plasmas with a Rf activity of at least up to 1200 IU/ml. By contrast, 21F12 and H31L21 showed a significant interference already at 300 IU/ml Rf activity. Furthermore, the appended Examples demonstrate that contrary to the antibody 21F12, the antibody of the present invention does not bind to IgM in natural samples with elevated Rf. The appended Examples also demonstrate that in a number of native samples with elevated Rf levels the prior art antibody 21F12 showed abnormally low results when compared to the levels detected with the new monoclonal antibody (clone 3.2.52). This demonstrates that the novel antibody provided herein can increase the accuracy of Aβ42 measurements in blood samples by avoiding abnormally low results of Aβ42 in a significant portion of native serum/plasma samples with elevated Rf levels.

Hence, in embodiments, the monoclonal antibody or the antigen-binding fragment of the invention when used as Aβ42 specific binding agent in an immunoassay (e.g. sandwich immunoassay) does not show rheumatoid factor (Rf) interference with a blood sample showing Rf interference with other Aβ42 specific binding agents (e.g. 21F12 and/or H31L21). The sample may have an Rf activity of 300 IU/ml or higher (preferably not higher than 1200 IU/ml). The predetermined amount of Aβ42 is selected in the measuring range of the respective immunoassay.

Rf interference preferably means that a detectable predetermined amount of Aβ42 in a sample with a Rf activity of 300 IU/ml or more (preferably not higher than 1200 IU/ml) is not correctly measured, i.e. is measured too high or too low (e.g. by at least 20% higher or lower than the predetermined amount). In a preferred embodiment, Rf interference means that a predetermined amount of Aβ42 in a sample with a Rf activity of 300 IU/ml or more (preferably not higher than 1200 IU/ml) is measured too low (e.g. 20% lower than the predetermined amount).

In embodiments, the monoclonal antibody or the antigen-binding fragment of the invention when used as Aβ42 specific binding agent in an immunoassay (e.g. sandwich immunoassay) detects the amount of Aβ42 in a blood sample, which shows Rf interference with other Aβ42 specific binding agents (e.g. 21F12 and/or H31L21) and comprises a detectable and predetermined Aβ42 amount, more accurately than other previously described Aβ42 antibodies (e.g. more accurately than 21F12 and/or H31L21) when used in an otherwise identical immunoassay. Preferably, the amount detected by the immunoassay using the antibody of the invention does not differ more than 20%, preferably more than 15% and even more preferably more than 10% from the predetermined amount.

The immunoassay as used in any of the Rf interference analysis may be a sandwich immunoassay, e.g. an Elecsys® immunoassay as described in the appended examples. The immunoassay may use an Aβ-specific binding agent specifically binding to any of amino acids 1 to 42 (i.e. Aβ specific but not Aβ42 specific) and not competing with binding to Aβ42 with the antibody or antigen-binding fragment of the invention as a second binding agent. Exemplary but not limiting Examples for such second binding agents are 3D6, 6E10 (BioLegend cat. no. 803004) and 1E8 (Nanotools cat. no. 0315-100/bA4N-1E8). In a preferred embodiment, the second binding agent is 3D6, as defined herein below.

In embodiments, a sample with an Rf activity may be a natural sample obtained from a subject suffering from rheumatoid arthritis. In alternative embodiments, the sample may be a sample spiked with a purified Rf concentrate to a final Rf activity corresponding to 300 IU/ml or higher (preferably not higher than 1200 IU/ml).

A “purified Rf concentrate” relates to Rfs (comprising Rf IgM) enriched from a pool of blood samples (e.g. plasma and/or serum). Methods for producing a “purified Rf concentrate” comprising Rf IgM are known in the art and are described in the appended Examples. In a preferred aspect, the purified Rf concentrate is obtained from a pool of plasma that has been obtained from rheumatoid arthritis patients. In embodiments, the purified Rf concentrate may be enriched for Rfs of type IgM.

In a preferred embodiment, the Rf concentrate (containing Rf IgM) may be obtained by a method as described by Tatum A H (J Immunol Methods. 1993 Jan. 14; 158(1):1-4. doi: 10.1016/0022-1759(93)90252-3). In particular, Rf concentrate (containing Rf IgM) may be obtained as described in the following and/or in the appended Examples. In brief, plasma from RA patients (e.g. verified to have Rf activity above 300 IU/ml) may be centrifuged to remove aggregated proteins. Next, the pH of supernatant may be adjusted to 4.75 using 2 M acetic acid and after incubation for 60 min at RT re-adjusted to 7.5 using 2 M Tris base. After centrifugation, the supernatant may be recalcified using 2.5 M CaCl₂×2H₂O (final conc 20 mM). After 2 h incubation at RT, the mixture may be centrifuged and the supernatant may be delipified by addition of 100 mL 3.53% dextrane sulfide, 4.47 M CaCl₂ solution per L plasma. After 30 min incubation at RT, the mixture may be centrifuged and the supernatant is to be kept. To enrich Rf IgMs, immunoglobulins may be precipitated using polyethylene glycol (PEG) and further purified via ammonium sulfate precipitation in order to eliminate residual PEG. The final pellet may then be dissolved in 50 mM potassium phosphate, 150 mM NaCl buffer. Finally, the Rf activity may be determined using Roche RF-II assay.

As the purified Rf concentrate is to be used as tool for determining Rf interference, the purified Rf concentrate may be tested to show a Rf interference in a reference Aβ42 immunoassay using the prior art antibodies 21F12 or H31L21 as Aβ42 specific binding agent. The reference immunoassay may be otherwise an immunoassay as described herein and in the appended Example. Particularly preferred is a sandwich immunoassay using 21F12 or H31L21 as a capture agent and 3D6 as a detection agent.

In embodiments, the monoclonal antibody or the antigen-binding fragment of the invention may, when used as binding agent in a sandwich immunoassay for quantifying Aβ42 in a blood sample, detect a predetermined amount of Aβ42 in a sample without Rf interference, said sample having an Rf activity between 300 IU/ml and 1200 IU/ml. The sample may be a sample spiked with a purified Rf concentrate, and said spiked samples. The sample may show an Rf interference in an otherwise identical sandwich immunoassay using the Aβ42 antibody 21F12 or H31L21 instead of the monoclonal antibody or the antigen-binding fragment of the invention. The immunoassay may be as defined herein elsewhere. For example, the immunoassay as used in any of the Rf interference analysis may be a sandwich immunoassay, e.g. an Elecsys® immunoassay as described in the appended examples. The immunoassay may use an Aβ-specific binding agent specifically binding to any of amino acids 1 to 42 (i.e. Aβ specific but not Aβ42 specific) and not competing with binding to Aβ42 with the antibody or antigen-binding fragment of the invention as a second binding agent. Exemplary but not limiting Examples for such second binding agents are mentioned, above.

The samples used for assessing Rf activity (whether natural or spiked with purified Rf concentrate) herein have an Rf activity of 300 IU/ml or higher. In embodiments, the Rf activity of said samples is 1200 IU/ml or lower. Accordingly, in preferred embodiments the Rf activity of the samples may be from 300 IU/ml to 1200 IU/ml. This Rf activity may be present naturally in the sample or may be spiked in using purified Rf concentrate. The Rf activity is preferably detected using the Roche RF-II assay (cat. no. 05480167190).

In additional or alternative embodiments, the monoclonal antibody or the antigen-binding fragment of the invention does not bind to one or more antibodies of subtype IgM when incubated with a blood sample having an Rf activity of 300 IU/ml or more (preferably not higher than 1200 IU/ml), said blood sample comprising antibodies of subtype IgM binding to the F(ab′)₂ region of the Aβ42 specific antibody 21F12 and/or to the F(ab′)₂ region of the Aβ42 specific antibody H31L21. In embodiments, the monoclonal antibody or the antigen-binding fragment of the invention does not bind to one or more antibodies of subtype IgM when incubated with a blood sample having an Rf activity of 300 IU/ml or more (preferably not higher than 1200 IU/ml), said blood sample comprising antibodies of subtype IgM binding to an F(ab′)2 region comprising the variable region of the Aβ1-42 antibody 21F12 and/or to an F(ab′)2 region comprising the variable region of the Aβ1-42 antibody H31L21.

“Does not bind” in this context means that the measured signal is below a certain cut-off value depending on the precise assay used. The cut-off level is determined using the same blood sample with a reference antibody, said antibody being known to be free of Rf interference. An exemplary reference antibody is an F(ab′)₂ fragment of the present invention having a heavy chain sequence of SEQ ID NO:9 and a light chain sequence of SEQ ID NO:10. Another, preferred reference antibody is TU1 (Roche Diagnostics; commercially available under Material number 10767778103).

As demonstrated by the appended Examples, the antibody or antigen-binding fragment of the invention specifically binding Aβ42 is characterized by excellent kinetic properties that make it ideally suitable for a use as reagent in an immunoassay for detecting and/or quantifying Aβ42 in samples.

In embodiments, the antibody or antigen-binding fragment of the invention binds Aβ42 at 37° C. with a dissociation equilibrium constant K_{D} of 20 nM or lower, preferably of 10 nM or lower and most preferably of 7 nM or lower. The K_{D} is preferably determined by surface plasmon resonance spectroscopy (e.g. Biacore).

In embodiments, the antibody or antigen-binding fragment of the invention binds Aβ42 at 37° C. with an association rate constant kₐ of 1*10⁴ M⁻¹s⁻¹ or higher, preferably 1*10⁵ M⁻¹s⁻¹ or higher, even more preferably 1*10⁶ M⁻¹s⁻¹ or higher, even more preferably 3*10⁶ M⁻¹s⁻¹ or higher and most preferably 3.8*10⁶ M⁻¹s⁻¹ or higher. A high association rate constant kₐ has the advantage that a fast binding of the antibody or antigen-binding fragment to Aβ42 is achieved. A fast binding in turn allows a shorter processing time for an immunoassay, as the incubation times can be kept short (e.g. 9 min as typically used for Elecsys®). The kₐ is preferably determined by surface plasmon resonance spectroscopy (e.g. Biacore).

In embodiments, the antibody or antigen-binding fragment of the invention binds Aβ42 at 37° C. with a dissociation rate constant k_{d} of 10⁻² s⁻¹ or lower, preferably 2*10⁻² s⁻¹ or lower and most preferably 2.5 10⁻² s⁻¹ or lower. A low dissociation rate constant ensures high complex stability with Aβ42. The k_{d} is preferably determined by surface plasmon resonance spectroscopy (e.g. Biacore).

In embodiments, the antibody/antigen complex half-life t/2 diss for the binding of the antibody or antigen-binding fragment of the invention to Aβ42 may be 30 sec or higher, preferably 45 sec or higher and most preferably 1 min or higher. The t/2 diss is preferably determined by surface plasmon resonance spectroscopy (e.g. Biacore).

The association rate constant kₐ [M⁻¹s⁻¹], the dissociation rate constant k_{d} [s⁻¹], the dissociation equilibrium constant K_{D} [M], and/or the antibody/antigen complex half-life t/2 diss can be measured with methods known in the art. In preferred embodiments, surface plasmon resonance spectroscopy (e.g. Biacore) may be employed. A non-limiting example for such methods is disclosed in the appended Examples.

The surface plasmon resonance spectroscopy may be calibrated such that the antibody 3.2.52 (Heavy chain sequence of SEQ ID NO: 23; and Light Chain sequence of SEQ ID NO: 10) shows the following measurement results: kₐ=3.8±0.1*10⁶ M⁻¹s⁻¹, k_{d}=2.53±0.06*10⁻² s⁻¹, t_{/2 diss}<1 min, MR (molar ratio)=0.7, K_{D}=6.7±0.2 nM.

In a non-limiting example, the measurement of the association rate constant kₐ [M⁻¹s⁻¹], the dissociation rate constant k_{d} [s⁻¹], the dissociation equilibrium constant K_{D} [M], and/or the antibody/antigen complex half-life t/2 diss may be performed using the BIAcore™ 8K+ instrument. Aβ42 concentration series ranging from 1.2 nM to 900 nM may be injected at 30 μL/min. The association phase may be monitored for 3 min, the dissociation phase between 5 min to 10 min at 37° C. The measurements may be performed using PBS-DT+, pH7.4 (10 mM phosphate buffer, 2.7 mM KCl, 137 mM NaCl. 5% DMSO, 0.05% Tween 20) as running buffer. The kinetic rate constants and the dissociation equilibrium constants K_{D} may be calculated using a Langmuir 1:1 fit model according Scrubber Version 2.0.c. The antibody/antigen complex half-life was calculated in minutes according to the formula t/2 diss=ln(2)/(k_{d}*60).

The measurements for determining the association rate constant kₐ [M⁻¹s⁻¹], the dissociation rate constant k_{d} [s⁻¹], the dissociation equilibrium constant K_{D} [M], and/or the antibody/antigen complex half-life t/2 diss may be conducted with Aβ42 or a peptide mimicking Aβ42. An Aβ42 mimicking peptide may comprise amino acids 1 to 12 and 35-42 of Aβ42, wherein amino acid 42 forms the C-terminus (e.g. amino acids 1 to 12 and 35-42 of Aβ42 fused with a linker). An exemplary but non-limiting Aβ42 mimicking peptide is Aβ(1-12)-O2Oc-O2Oc-Lys(Bi)-O2Oc-O2Oc-Aβ(35-42) (also termed as amino acids 1-12 and 35-42 (connected with a linker) of the Aβ42 peptide in the appended Examples).

In embodiments, the monoclonal antibody or an antigen-binding fragment specifically binding to Aβ42 provided herein comprises a heavy chain variable domain (VH) comprising (a) a CDR-H1 comprising the amino acid sequence of SEQ ID NO: 3 or a variant thereof with one amino acid substitution, (b) a CDR-H2 comprising the amino acid sequence of SEQ ID NO: 4 or a variant thereof with one amino acid substitution, and (c) a CDR-H3 comprising the amino acid sequence of SEQ ID NO: 5 or a variant thereof with one amino acid substitution, and wherein the light chain variable domain (VL) comprises (d) a CDR-L1 comprising the amino acid sequence of SEQ ID NO: 6 or a variant thereof with one amino acid substitution, (e) a CDR-L2 comprising the amino acid sequence of SEQ ID NO: 7 or a variant thereof with one amino acid substitution, and (f) a CDR-L3 comprising the amino acid sequence of SEQ ID NO: 8 or a variant thereof with one amino acid substitution.

In embodiments, the monoclonal antibody or an antigen-binding fragment specifically binding to Aβ42 provided herein comprises a heavy chain variable domain (VH) comprising (a) a CDR-H1 consisting of the amino acid sequence of SEQ ID NO: 3 or a variant thereof with one amino acid substitution, (b) a CDR-H2 consisting of the amino acid sequence of SEQ ID NO: 4 or a variant thereof with one amino acid substitution, and (c) a CDR-H3 consisting of the amino acid sequence of SEQ ID NO: 5 or a variant thereof with one amino acid substitution, and wherein the light chain variable domain (VL) comprises (d) a CDR-L1 consisting of the amino acid sequence of SEQ ID NO: 6 or a variant thereof with one amino acid substitution, (e) a CDR-L2 consisting of the amino acid sequence of SEQ ID NO: 7 or a variant thereof with one amino acid substitution, and (f) a CDR-L3 consisting of the amino acid sequence of SEQ ID NO: 8 or a variant thereof with one amino acid substitution.

In embodiments, the one amino acid substitution(s) in the CDR-H1, CDR-H2, CDR-H3, CDR-L1, CDR-L2 and/or CDR-L3 are conservative amino acid exchanges.

Accordingly, in embodiments, the monoclonal antibody or an antigen-binding fragment specifically binding to Aβ42 provided herein comprises a heavy chain variable domain (VH) comprising (a) a CDR-H1 comprising the amino acid sequence of SEQ ID NO: 3 or a variant thereof with one conservative amino acid substitution, (b) a CDR-H2 comprising the amino acid sequence of SEQ ID NO: 4 or a variant thereof with one conservative amino acid substitution, and (c) a CDR-H3 comprising the amino acid sequence of SEQ ID NO: 5 or a variant thereof with one conservative amino acid substitution, and wherein the light chain variable domain (VL) comprises (d) a CDR-L1 comprising the amino acid sequence of SEQ ID NO: 6 or a variant thereof with one conservative amino acid substitution, (e) a CDR-L2 comprising the amino acid sequence of SEQ ID NO: 7 or a variant thereof with one conservative amino acid substitution, and (f) a CDR-L3 comprising the amino acid sequence of SEQ ID NO: 8 or a variant thereof with one conservative amino acid substitution.

In specific embodiments, the monoclonal antibody or an antigen-binding fragment specifically binding to Aβ42 provided herein comprises a heavy chain variable domain (VH) comprising (a) a CDR-H1 consisting of the amino acid sequence of SEQ ID NO: 3 or a variant thereof with one conservative amino acid substitution, (b) a CDR-H2 consisting of the amino acid sequence of SEQ ID NO: 4 or a variant thereof with one conservative amino acid substitution, and (c) a CDR-H3 consisting of the amino acid sequence of SEQ ID NO: 5 or a variant thereof with one conservative amino acid substitution, and wherein the light chain variable domain (VL) comprises (d) a CDR-L1 consisting of the amino acid sequence of SEQ ID NO: 6 or a variant thereof with one conservative amino acid substitution, (e) a CDR-L2 consisting of the amino acid sequence of SEQ ID NO: 7 or a variant thereof with one conservative amino acid substitution, and (f) a CDR-L3 consisting of the amino acid sequence of SEQ ID NO: 8 or a variant thereof with one conservative amino acid substitution.

In embodiments, the one amino acid substitution(s) in the CDR-H1, CDR-H2, CDR-H3, CDR-L1, CDR-L2 and/or CDR-L3 are highly conservative amino acid exchanges.

Accordingly, in embodiments, the monoclonal antibody or an antigen-binding fragment specifically binding to Aβ42 provided herein comprises a heavy chain variable domain (VH) comprising (a) a CDR-H1 comprising the amino acid sequence of SEQ ID NO: 3 or a variant thereof with one highly conservative amino acid substitution, (b) a CDR-H2 comprising the amino acid sequence of SEQ ID NO: 4 or a variant thereof with one highly conservative amino acid substitution, and (c) a CDR-H3 comprising the amino acid sequence of SEQ ID NO: 5 or a variant thereof with one highly conservative amino acid substitution, and wherein the light chain variable domain (VL) comprises (d) a CDR-L1 comprising the amino acid sequence of SEQ ID NO: 6 or a variant thereof with one highly conservative amino acid substitution, (e) a CDR-L2 comprising the amino acid sequence of SEQ ID NO: 7 or a variant thereof with one highly conservative amino acid substitution, and (f) a CDR-L3 comprising the amino acid sequence of SEQ ID NO: 8 or a variant thereof with one highly conservative amino acid substitution.

In specific embodiments, the monoclonal antibody or an antigen-binding fragment specifically binding to Aβ42 provided herein comprises a heavy chain variable domain (VH) comprising (a) a CDR-H1 consisting of the amino acid sequence of SEQ ID NO: 3 or a variant thereof with one highly conservative amino acid substitution, (b) a CDR-H2 consisting of the amino acid sequence of SEQ ID NO: 4 or a variant thereof with one highly conservative amino acid substitution, and (c) a CDR-H3 consisting of the amino acid sequence of SEQ ID NO: 5 or a variant thereof with one highly conservative amino acid substitution, and wherein the light chain variable domain (VL) comprises (d) a CDR-L1 consisting of the amino acid sequence of SEQ ID NO: 6 or a variant thereof with one highly conservative amino acid substitution, (e) a CDR-L2 consisting of the amino acid sequence of SEQ ID NO: 7 or a variant thereof with one highly conservative amino acid substitution, and (f) a CDR-L3 consisting of the amino acid sequence of SEQ ID NO: 8 or a variant thereof with one highly conservative amino acid substitution.

As explained in the appended Examples, there is strong evidence that the Rf interference observed with the prior art antibody 21F12 is caused by its VH and VL domain, more specifically likely by its CDR residues. The VH and VL regions of 21F12 and 3.2.52 (an exemplary antibody of the invention) show only minimal differences in the framework regions, as evident from the sequence alignments shown in FIGS. 7 and 8. Sequence differences in the VH framework regions (i.e. outside the CDRs as defined by Kabat) of 3.2.52 and 21F12 are only found in the amino acids corresponding to positions 12, 19, 20, 23, 24, 29, 37, 72, 77, 79, 82, 95, 97, 113, 114 and 118 of SEQ ID NO:1. These positions correspond to positions 12, 19, 20, 23, 24, 29, 37, 71, 76, 78, 81, 91, 93, 108, 109 and 113 according to Kabat nomenclature. Sequence differences in the VL framework regions (i.e. outside the CDRs as defined by Kabat) of 3.2.52 and 21F12 are only found in the amino acids corresponding to positions 10, 12, 14, 15, 17, 18, 19, 41, 42, 44, 51, 74, 92, 105, 110, 111 and 112 of SEQ ID NO:2. These positions correspond to positions 10, 12, 14, 15, 17, 18, 19, 36, 37, 39, 46, 69, 87, 100, 105, 106 and 107 according to Kabat nomenclature.

Accordingly, in embodiments, the VH of the monoclonal antibody or antigen-binding fragment of the invention comprises the CDRs as defined in any of the embodiments herein above, and further comprises the amino acids at positions 12, 19, 20, 23, 24, 29, 37, 72, 77, 79, 82, 95, 97, 113, 114 and 118 of SEQ ID NO: 1 at the positions corresponding thereto.

In embodiments, the VH of the monoclonal antibody or antigen-binding fragment of the invention comprises the CDRs as defined in any of the embodiments herein above, and further comprises the amino acids at positions 12, 19, 20, 23, 24, 29, 37, 72, 77, 79, 82, 95, 97, 113, 114 and 118 of SEQ ID NO: 1 at the positions 12, 19, 20, 23, 24, 29, 37, 71, 76, 78, 81, 91, 93, 108, 109 and 113 according to Kabat nomenclature, respectively.

In embodiments, the VH of the monoclonal antibody or antigen-binding fragment of the invention comprises the CDRs as defined in any of the embodiments herein above, and further comprises the amino acids of SEQ ID NO: 1 corresponding to positions 12, 19, 20, 23, 24, 29, 37, 71, 76, 78, 81, 91, 93, 108, 109 and 113 according to Kabat nomenclature.

In embodiments, the VL of the monoclonal antibody or antigen-binding fragment of the invention comprises the CDRs as defined in any of the embodiments herein above, and further comprises the amino acids at positions 10, 12, 14, 15, 17, 18, 19, 41, 42, 44, 51, 74, 92, 105, 110, 111 and 112 of SEQ ID NO: 2 at the positions corresponding thereto.

In embodiments, the VL of the monoclonal antibody or antigen-binding fragment of the invention comprises the CDRs as defined in any of the embodiments herein above, and further comprises the amino acids at positions 10, 12, 14, 15, 17, 18, 19, 41, 42, 44, 51, 74, 92, 105, 110, 111 and 112 of SEQ ID NO:2 at the positions 10, 12, 14, 15, 17, 18, 19, 36, 37, 39, 46, 69, 87, 100, 105, 106 and 107 according to Kabat nomenclature, respectively.

In embodiments, the VL of the monoclonal antibody or antigen-binding fragment of the invention comprises the CDRs as defined in any of the embodiments herein above, and further comprises the amino acids of SEQ ID NO:2 corresponding to positions 10, 12, 14, 15, 17, 18, 19, 36, 37, 39, 46, 69, 87, 100, 105, 106 and 107 according to Kabat nomenclature.

In embodiments, the monoclonal antibody or an antigen-binding fragment of the first aspect comprises:
(i) a heavy chain variable domain (VH) comprising the amino acid sequence of SEQ ID NO: 1 or an amino acid sequence having a sequence identity of at least 70%, preferably 80%, even more preferably 90%, even more preferably 95% and most preferably 99% with SEQ ID NO: 1; and
(ii) a light chain variable domain (VL) comprising the amino acid sequence of SEQ ID NO: 2 or an amino acid sequence having a sequence identity of at least 70%, preferably 80%, even more preferably 90%, even more preferably 95% and most preferably 99% with SEQ ID NO: 2.

In specific embodiments, the monoclonal antibody or an antigen-binding fragment of the first aspect comprises:
(i) a heavy chain variable domain (VH) consisting of the amino acid sequence of SEQ ID NO: 1 or an amino acid sequence having a sequence identity of at least 70%, preferably 80%, even more preferably 90%, even more preferably 95% and most preferably 99% with SEQ ID NO: 1; and
(ii) a light chain variable domain (VL) consisting of the amino acid sequence of SEQ ID NO: 2 or an amino acid sequence having a sequence identity of at least 70%, preferably 80%, even more preferably 90%, even more preferably 95% and most preferably 99% with SEQ ID NO: 2.

In embodiments, the monoclonal antibody or an antigen-binding fragment of the first aspect comprises a heavy chain variable domain (VH) comprising the amino acid sequence of SEQ ID NO: 1; and a light chain variable domain (VL) comprising the amino acid sequence of SEQ ID NO: 2.

In specific embodiments, the monoclonal antibody or an antigen-binding fragment of the first aspect comprises a heavy chain variable domain (VH) consisting of the amino acid sequence of SEQ ID NO: 1; and a light chain variable domain (VL) consisting of the amino acid sequence of SEQ ID NO: 2.

In embodiments, the monoclonal antibody or an antigen-binding fragment specifically binding to Aβ42 provided herein comprises a heavy chain variable domain (VH) comprising (a) a CDR-H1 as comprised in the amino acid sequence of SEQ ID NO: 1, (b) a CDR-H2 as comprised in the amino acid sequence of SEQ ID NO: 1, and (c) a CDR-H3 as comprised in the amino acid sequence of SEQ ID NO: 1, and wherein the light chain variable domain (VL) comprises (d) a CDR-L1 as comprised in the amino acid sequence of SEQ ID NO: 2, (e) a CDR-L2 as comprised in the amino acid sequence of SEQ ID NO: 2, and (f) as comprised in the amino acid sequence of SEQ ID NO: 2.

In this context, in principle any CDR definition methods as known in the art and/or as described herein elsewhere may be employed. In preferred embodiments, the CDRs are defined according to Kabat nomenclature.

Variable regions/domains/sequences of both heavy and light chains of antibodies comprise the CDRs, determining the specificity of the antibodies, and the more generic framework regions.

A light chain variable domain/sequence consists of framework regions (FWs) and CDRs as represented in formula I:

FW(LC)1-CDR(LC)1-FW(LC)2-CDR(LC)2-FW(LC)3-CDR(LC)3-FW(LC)4 (formula I).

A heavy chain variable domain/sequence consists of FWs and CDRs as represented in formula II:

FW(HC)1-CDR(HC)1-FW(HC)2-CDR(HC)2-FW(HC)3-CDR(HC)3-FW(HC)4 (formula II).

The primary structure shown in formula I represents the order of the components of the light chain variable domain of the antibody of the present invention from the N-terminus to the C-terminus. The primary structure shown in formula II represents the order of the components of the heavy chain variable domain of the antibody of the present invention from the N-terminus to the C-terminus. In each case, framework region (FW) 1 represents the most N-terminal part of the respective variable chain domain, while FW 4 represents the most C-terminal part of the respective variable chain domain.

The skilled artisan can perfectly deduce the sequence of each FW-region once, as in the present disclosure, the full-length variable chain sequence and the sequences of the CDRs comprised therein are given.

As will be appreciated by the skilled artisan a certain degree of sequence variation the framework regions will not affect the antibodies function and features as described herein.

In embodiments, the VH of the monoclonal antibody or antigen-binding fragment thereof specifically binding to Aβ42 according to the invention may comprise
(i) the FW(HC)1 as comprised in SEQ ID NO: 1 or a variant thereof having a sequence identity of at least 70%, preferably 80%, even more preferably 90%, even more preferably 95% and most preferably 99% to the FW(HC)1 as comprised in SEQ ID NO: 1;
(ii) the FW(HC)2 as comprised in SEQ ID NO: 1 or a variant thereof having a sequence identity of at least 70%, preferably 80%, even more preferably 90%, even more preferably 95% and most preferably 99% to the FW(HC)2 as comprised in SEQ ID NO: 1;
(iii) the FW(HC)3 as comprised in SEQ ID NO: 1 or a variant thereof having a sequence identity of at least 70%, preferably 80%, even more preferably 90%, even more preferably 95% and most preferably 99% to the FW(HC)3 as comprised in SEQ ID NO: 1; and
(iv) the FW(HC)4 as comprised in SEQ ID NO: 1 or a variant thereof having a sequence identity of at least 70%, preferably 80%, even more preferably 90%, even more preferably 95% and most preferably 99% to the FW(HC)4 as comprised in SEQ ID NO: 1.

In embodiments, the VL of the monoclonal antibody or antigen-binding fragment thereof specifically binding to Aβ42 according to the invention may comprise
(i) the FW(LC)1 as comprised in SEQ ID NO: 2 or a variant thereof having a sequence identity of at least 70%, preferably 80%, even more preferably 90%, even more preferably 95% and most preferably 99% to the FW(LC)1 as comprised in SEQ ID NO: 2;
(ii) the FW(LC)2 as comprised in SEQ ID NO: 2 or a variant thereof having a sequence identity of at least 70%, preferably 80%, even more preferably 90%, even more preferably 95% and most preferably 99% to the FW(LC)2 as comprised in SEQ ID NO: 2;
(iii) the FW(LC)3 as comprised in SEQ ID NO: 2 or a variant thereof having a sequence identity of at least 70%, preferably 80%, even more preferably 90%, even more preferably 95% and most preferably 99% to the FW(LC)3 as comprised in SEQ ID NO: 2; and
(iv) the FW(LC)4 as comprised in SEQ ID NO: 2 or a variant thereof having a sequence identity of at least 70%, preferably 80%, even more preferably 90%, even more preferably 95% and most preferably 99% to the FW(LC)4 as comprised in SEQ ID NO: 2.

In embodiments, the VH of the monoclonal antibody or antigen-binding fragment thereof specifically binding to Aβ42 according to the invention may comprise
(i) the FW(HC)1 of SEQ ID NO: 11 or a variant thereof having a sequence identity of at least 70%, preferably 80%, even more preferably 90%, even more preferably 95% and most preferably 99% thereto;
(ii) the FW(HC)2 as comprised in SEQ ID NO: 12 or a variant thereof having a sequence identity of at least 70%, preferably 80%, even more preferably 90%, even more preferably 95% and most preferably 99% thereto;
(iii) the FW(HC)3 as comprised in SEQ ID NO: 13 or a variant thereof having a sequence identity of at least 70%, preferably 80%, even more preferably 90%, even more preferably 95% and most preferably 99% thereto; and
(iv) the FW(HC)4 as comprised in SEQ ID NO: 14 or a variant thereof having a sequence identity of at least 70%, preferably 80%, even more preferably 90%, even more preferably 95% and most preferably 99% thereto.

In embodiments, the VL of the monoclonal antibody or antigen-binding fragment thereof specifically binding to Aβ42 according to the invention may comprise
(i) the FW(LC)1 as comprised in SEQ ID NO: 15 or a variant thereof having a sequence identity of at least 70%, preferably 80%, even more preferably 90%, even more preferably 95% and most preferably 99% thereto;
(ii) the FW(LC)2 as comprised in SEQ ID NO: 16 or a variant thereof having a sequence identity of at least 70%, preferably 80%, even more preferably 90%, even more preferably 95% and most preferably 99% thereto;
(iii) the FW(LC)3 as comprised in SEQ ID NO: 17 or a variant thereof having a sequence identity of at least 70%, preferably 80%, even more preferably 90%, even more preferably 95% and most preferably 99% thereto; and
(iv) the FW(LC)4 as comprised in SEQ ID NO: 18 or a variant thereof having a sequence identity of at least 70%, preferably 80%, even more preferably 90%, even more preferably 95% and most preferably 99% thereto.

The above described degree of variation in the framework regions as compared to the respective specifically recited amino acid sequence can be due to the substitution, insertion, addition, and/or deletion of (an) amino acid(s). The variants of the FW are limited to such variations that are still functional, i.e. specifically bind Aβ42, do show reduced or no Rf interference as observed for prior art antibodies and show when being used in an immunoassay the at least the same clinical performance as antibody 3.2.52 as described herein.

In embodiments, the variation in the amino acid sequences of the framework regions is due to the substitution of (an) amino acid(s). Substitutions can be conservative amino acid substitutions (or highly conservative amino acid substitutions) or non-conservative amino acid substitutions.

In embodiments, each of the substitutions in the framework region compared to the respective specifically recited amino acid sequence may be a conservative substitution.

In specific embodiments, each of the substitutions in the framework region with respect to the indicated sequences may be a highly conservative substitution.

In embodiments, the monoclonal antibody or an antigen-binding fragment of the first aspect comprises:
(i) a heavy chain comprising the amino acid sequence of SEQ ID NO: 9 or an amino acid sequence having a sequence identity of at least 70%, preferably 80%, even more preferably 90%, even more preferably 95% and most preferably 99% with SEQ ID NO: 9; and
(ii) a light chain comprising the amino acid sequence of SEQ ID NO: 10 or an amino acid sequence having a sequence identity of at least 70%, preferably 80%, even more preferably 90%, even more preferably 95% and most preferably 99% with SEQ ID NO: 10.

In specific embodiments, the monoclonal antibody or an antigen-binding fragment of the first aspect comprises:
(i) a heavy chain consisting of the amino acid sequence of SEQ ID NO: 9 or an amino acid sequence having a sequence identity of at least 70%, preferably 80%, even more preferably 90%, even more preferably 95% and most preferably 99% with SEQ ID NO: 9; and
(ii) a light chain consisting of the amino acid sequence of SEQ ID NO: 10 or an amino acid sequence having a sequence identity of at least 70%, preferably 80%, even more preferably 90%, even more preferably 95% and most preferably 99% with SEQ ID NO: 10.

In embodiments, the monoclonal antibody or an antigen-binding fragment of the first aspect comprises a heavy chain comprising the amino acid sequence of SEQ ID NO: 9; and a light chain comprising the amino acid sequence of SEQ ID NO: 10.

In specific embodiments, the monoclonal antibody or an antigen-binding fragment of the first aspect comprises a heavy chain consisting of the amino acid sequence of SEQ ID NO: 9; and a light chain consisting of the amino acid sequence of SEQ ID NO: 10.

In embodiments, the monoclonal antibody or antigen-binding fragment is an F(ab′)₂ fragment.

A skilled person in the art can generate antibodies or antigen-binding fragments with similar or improved affinity to Aβ42 using affinity maturation methods known in the art. Accordingly, provided herein are also variants of any of the antibodies or antigen-binding fragments specifically binding to Aβ42 as defined herein obtained by affinity maturation. An “affinity matured” antibody refers to an antibody with one or more alterations in one or more complementary determining regions (CDRs), compared to a parent antibody, which does not possess such alterations, such alterations resulting in an improvement in the affinity of the antibody for antigen. In embodiments, the affinity maturated antibody has at most one amino acid substitution, insertion and/or deletion per CDR.

Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Examples for screening method to identify antibody variants are a phage display technology or a ribosome display technology.

The monoclonal antibody or antigen-binding fragment of the invention may be a mouse antibody. The monoclonal antibody or antigen fragment may be of any IgG subclass. A mouse monoclonal antibody or antigen-binding fragment of the invention may be of subclass IgG1, IgG2a/c, IgG2b or IgG3. In embodiments, a mouse monoclonal antibody or antigen-binding fragment of the invention may be of subclass IgG2b. In embodiments, the monoclonal antibody or antigen-binding fragment of the invention may be comprise the sequences of mouse subclass IgG2b.

The monoclonal antibody or antigen-binding fragment according the first aspect is in embodiments, for use in an assay (e.g. in vitro) for detecting or quantifying Aβ42 in a sample.

In embodiments, the monoclonal antibody or antigen-binding fragment may have a label attached thereto. The label may be a detection label. The label may be a capture label.

The monoclonal antibody or antigen binding fragment having attached thereto a capture label may be for use as a capture agent, e.g. in a heterogeneous immunoassay (e.g. sandwich immunoassay).

The monoclonal antibody or antigen binding fragment having attached thereto a detection label may be for use as a detection agent, e.g. in a heterogeneous immunoassay (e.g. sandwich immunoassay).

A detection label includes in principle any functional moiety not naturally forming part of the antibody allowing for the detection of the antibody or the antigen-binding fragment thereof. Non-limiting examples for groups and individual detection labels are disclosed herein below.

A capture label is a label which mediates or allows binding of the antibody or antigen-fragment to a solid surface, such as microbeads (e.g. magnetic microbeads). In embodiments, the capture label may be a member of a binding pair which can interact with another member of the binding pair (said other member being e.g. coated on the solid surface). Non-limiting examples for binding pairs are biotin-streptavidin, a pair of hybridizing oligo- or polynucleotides or analogs thereof capable of forming with each other a duplex, biotin-(strept)avidin, antibody-hapten, antibody-antigen, enzyme-substrate, [mannose, maltose, amylose]-[respective sugar-binding protein], [oligo- or polysaccharide]-lectin, cytokine-[respective receptor] or ligand-[respective ligand-binding domain], [Zn²⁺, Ni²⁺, Co²⁺, or Cu²⁺ metal-chelate complex]-[histidine-tag], [indium chelate complex]-[CHA255 antibody], [cucurbit[n]uril host residue]-[guest residue], [first protein dimerization domain]-[second protein dimerization domain].

In one embodiment, the monoclonal Aβ42 specific antibody of the invention is an isolated monoclonal antibody. An “isolated” antibody or antigen binding fragment thereof, is one which has been identified and separated and/or recovered from a component of its natural environment. Contaminant components of its natural environment are materials which would interfere with research, diagnostic or therapeutic uses for the specific bind agent, e.g. an antibody, and may include enzymes, hormones, and other proteinaceous or non-proteinaceous solutes. In some embodiments, a specific binding agent is purified (1) to greater than 90% by weight as determined by, for example, the Lowry method, and in some embodiments, to greater than 95% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Ordinarily, an isolated specific binding agent, e.g. an isolated antibody will be prepared by at least one purification step e.g. using protein purification techniques well known in the art.

In a second aspect, the present invention also provides a nucleic acid molecule, or a set of nucleic acid molecules (e.g. a first and a second) encoding the monoclonal antibody of the invention or any antigen-binding fragments thereof, as defined herein above. In particular provided is a polynucleotide encoding the heavy chain and/or light chain, or the heavy chain variable domain and/or light chain variable domain of the monoclonal antibody specifically binding to Aβ42 as defined herein above. In some embodiments, the polynucleotide may comprise further sequences to ensure that not only the heavy and/or light chain variable domain are expressed, but also the remaining heavy and/or light chain constant regions such that a full-length IgG antibody is expressed comprising the heavy and light chain variable domains of the invention. Accordingly, for each of the embodiments relating to monoclonal antibodies or antigen binding fragments specifically binding Aβ42 as described herein a corresponding polynucleotide or set of polynucleotides encoding the respective antibody or antigen binding fragment is provided.

The heavy chain and light chain or the heavy chain variable region and light chain region of the monoclonal antibody or antigen-binding fragment thereof may be encoded on a single nucleic acid or on a set of a first and second polynucleotide (the first polynucleotide encoding the heavy chain or heavy chain variable region, the second polynucleotide encoding the light chain or light chain variable region.

The nucleic acid molecules of the invention can e.g. be synthesized by standard chemical synthesis methods and/or recombinant methods, or produced semi-synthetically, e.g. by combining chemical synthesis and recombinant methods. Ligation of the coding sequences to transcriptional regulatory elements and/or to other amino acid encoding sequences can be carried out using established methods, such as restriction digests, ligations and molecular cloning.

In a third aspect, provided herein is a vector that comprises a polynucleotide or a set of polynucleotides of the second aspect. In particular, provided are vectors comprising a nucleic acid molecule encoding an antibody or antibody antigen binding fragment of the invention. As used herein, the term “vector” relates to a circular or linear nucleic acid molecule that can autonomously replicate in a host cell into which it has been introduced. Non-limiting examples of vectors suitable for use in the present invention include cosmids, plasmids (e.g., naked or contained in liposomes), viruses (e.g., lentiviruses, retroviruses, adenoviruses, and adeno-associated viruses) and bacteriophages. However, the art provides many suitable vectors, the choice of which depends on the desired function. The development and use of suitable vectors is well documented in the art; see, for example, the techniques described in Sambrook and Russel “Molecular Cloning, A Laboratory Manual”, Cold Spring Harbor Laboratory, N.Y. (2001) and Ausubel, “Current Protocols in Molecular Biology”, Green Publishing Associates and Wiley Interscience, N.Y. (1989), (1994). Vectors of use in connection with the present invention comprise a nucleic acid sequence encoding the full length anti-aβ42 antibody or antigen binding fragment thereof as disclosed herein. As such, for each of the embodiments relating to monoclonal antibodies or antigen binding fragments specifically binding Aβ42 as described herein a vector comprising the corresponding polynucleotide encoding the respective antibody or antigen binding fragment is provided.

With regard to the term “vector comprising” as used herein, it is understood in the art that further nucleic acid sequences are present in the vectors that are necessary and/or sufficient for desired vector activity in the host cell, e.g. drive replication of the vector (and, thus the encoding nucleic acid sequences) and/or to direct the host cell express the antibody or antigen binding fragment of the invention. Such further nucleic acid sequences include but are not limited to sequences controlling vector replication and/or expression of a desired sequence in the particular cell system. For example, the vectors may comprise the nucleic acid molecule encoding an antibody or antibody antigen binding fragment of the invention operably linked and/or under the control of regulatory sequences. The term “regulatory sequence” refers to DNA sequences that are necessary to effect the expression of coding sequences to which they are operably linked. The term “control sequence” is intended to include, at a minimum, all components the presence of which may also be necessary for expression, and may further include additional advantageous components, e.g., to allow replication. As is understood in the art, the nature of such regulatory and control sequences differs depending upon the host organism. For example, in prokaryotes, control sequences generally include promoters, ribosomal binding sites, and terminators. In eukaryotes control sequences generally include promoters, terminators and, in some instances, enhancers, transactivators and/or transcription factors.

The vectors of use in the present invention are preferably expression vectors. An expression vector is capable of directing the replication and the expression of the nucleic acid molecule of the invention in a host cell and, accordingly, provides for the expression of, e.g., the heavy chain and/or light chain variable domains or the heavy chain and/or light chain of the monoclonal antibodies specifically binding to Aβ42 as disclosed herein. In some embodiments, the vector may comprise further sequences to ensure that not only the heavy and light chain variable domains are expressed, but also the remaining heavy and light chain constant regions such that a full-length IgG antibody or an antibody fragment such as a F(ab)2 fragment is expressed comprising the heavy and light chain variable domains of the invention. Suitable expression vectors have been widely described in the literature and the determination of the appropriate expression vector for a particular cell system can be readily made by the skilled person using routine methods. Preferably, the vectors disclosed herein comprise a recombinant polynucleotide (i.e., a nucleic acid sequence encoding the monoclonal antibody according to the invention) as well as expression operably linked control sequences. The vectors as provided herein preferably further comprise a promoter. The herein described vectors may also comprise a selection marker gene and a replication-origin ensuring replication in the host Moreover, the herein provided vectors may also comprise a termination signal for transcription. Expression vectors as known in the art may drive transient or constitutive expression in a host cell.

Non-limiting examples of vectors include pQE-12, the pUC-series, pBluescript (Stratagene), the pET-series of expression vectors (Novagen) or pCRTOPO (Invitrogen), lambda gt11, pJOE, the pBBR1-MCS series, pJB861, pBSMuL, pBC2, pUCPKS, pTACT1, pTRE, pCAL-n-EK, pESP-1, pOP13CAT, the E-027 pCAG Kosak-Cherry (L45a) vector system, pREP (Invitrogen), pCEP4 (Invitrogen), pMClneo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, Okayama-Berg cDNA expression vector pcDV1 (Pharmacia), pRc/CMV, pcDNA1, pcDNAβ (Invitrogen), pcDNA3.1, pSPORT1 (GIBCO BRL), pGEMHE (Promega), pLXIN, pSIR (Clontech), pIRES-EGFP (Clontech), pEAK-10 (Edge Biosystems) pTriEx-Hygro (Novagen) and pCINeo (Promega). Non-limiting examples for plasmid vectors suitable for Pichia pastoris comprise e.g. the plasmids pAO815, pPIC9K and pPIC3.5K (all Invitrogen). Another vector suitable for expressing proteins in Xenopus embryos, zebrafish embryos as well as a wide variety of mammalian and avian cells is the multipurpose expression vector pCS2+.

Generally, vectors can contain one or more origins of replication (ori) and inheritance systems for cloning or expression, one or more markers for selection in the host, e.g., antibiotic resistance, and one or more expression cassettes. In addition, the coding sequences comprised in the vector can be ligated to transcriptional regulatory elements and/or to other amino acid encoding sequences using established methods. Such regulatory sequences are well known to those skilled in the art and include, without being limiting, regulatory sequences ensuring the initiation of transcription, internal ribosomal entry sites (IRES) (Owens, G. C. et al. [2001] Proc. Natl. Acad. Sci. U.S.A. 98:1471-1476) and optionally regulatory elements ensuring termination of transcription and stabilization of the transcript. Non-limiting examples for such regulatory elements ensuring the initiation of transcription comprise promoters, a translation initiation codon, enhancers, insulators and/or regulatory elements ensuring transcription termination, which are to be included downstream of the nucleic acid molecules of the invention. Further examples include Kozak sequences and intervening sequences flanked by donor and acceptor sites for RNA splicing, nucleotide sequences encoding secretion signals or, depending on the expression system used, signal sequences capable of directing the expressed protein to a cellular compartment or to the culture medium. The vectors may also contain an additional expressible polynucleotide coding for one or more chaperones to facilitate correct protein folding.

Additional examples of suitable origins of replication include, for example, the full length ColE1, a truncated ColE1, the SV40 viral and the M13 origins of replication, while additional examples of suitable promoters include, without being limiting, the cytomegalovirus (CMV) promoter, SV40-promoter, RSV-promoter (Rous sarcome virus), the lacZ promoter, the tetracycline promoter/operator (tet^{P/o}), chicken β-actin promoter, CAG-promoter (a combination of chicken β-actin promoter and cytomegalovirus immediate-early enhancer), the gai10 promoter, human elongation factor 1α-promoter, AOX1 promoter, GAL1 promoter CaM-kinase promoter, the lac, trp or tac promoter, the T7 or T5 promoter, the lacUV5 promoter, the Autographa californica multiple nuclear polyhedrosis virus (AcMNPV) polyhedral promoter or a globin intron in mammalian and other animal cells. One example of an enhancer is e.g. the SV40-enhancer. Non-limiting additional examples for regulatory elements ensuring transcription termination include the SV40-poly-A site, the tk-poly-A site, the rho-independent lpp terminator or the AcMNPV polyhedral polyadenylation signals. Further non-limiting examples of selectable markers include dhfr, which confers resistance to methotrexate (Reiss, Plant Physiol. (Life Sci. Adv.) 13 (1994), 143-149), npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to blasticidin S (Tamura, Biosci. Biotechnol. Biochem. 59 (1995), 2336-2338).

In a further embodiment, the vector is a eukaryotic expression plasmid containing an expression cassette consisting of a 5′ CMV promoter including Intron A, and a 3′ BGH polyadenylation sequence. In addition to the expression cassette, the plasmid can contain a pUC18-derived origin of replication and a beta-lactamase gene conferring ampicillin resistance for plasmid amplification in E. coli. For secretion of the antibodies, a eukaryotic leader sequence can be cloned 5′ of the antibody gene.

The nucleic acid molecules and/or vectors of the invention can be designed for transfection into prokaryotic or eukaryotic host cells by any means known in the art or described herein. Non-limiting examples of suitable methods include chemical based methods (polyethylenimine, calcium phosphate, liposomes, DEAE-dextrane, nucleofection), nonchemical methods (electroporation, sonoporation, optical transfection, gene electrotransfer, hydrodynamic delivery or naturally occurring transformation upon contacting cells with the nucleic acid molecule of the invention), particle-based methods (gene gun, magnetofection, impalefection) phage vector-based methods and viral methods. For example, expression vectors derived from viruses such as retroviruses, vaccinia virus, adeno-associated virus, herpes viruses, Semliki Forest Virus or bovine papilloma virus, may be used for transfection of the nucleic acid molecules into targeted cell population. Additionally, baculoviral systems can also be used as vector in eukaryotic expression system for the nucleic acid molecules of the invention.

Hence, in a fourth aspect the present invention relates to a host cell comprising a polynucleotide according to the invention, or a vector according to the invention. The host cell may be a prokaryotic cell or a eukaryotic cell.

The term “prokaryote” is meant to include all bacteria which can be transformed, transduced or transfected with DNA or DNA or RNA molecules for the expression of a protein of the invention. Prokaryotic hosts may include gram negative as well as gram positive bacteria such as, for example, E. coli, S. typhimurium, Serratia marcescens, Corynebacterium (glutamicum), Pseudomonas (fluorescens), Lactobacillus, Streptomyces, Salmonella and Bacillus subtilis.

The term “eukaryotic” is meant to include yeast, higher plant, insect and mammalian cells. Non-limiting examples of mammalian host cells typically used in the art include, Hela, HEK293, H9, Per.C6 and Jurkat cells, mouse NIH3T3, NS/0, SP2/0 and C127 cells, COS cells, e.g. COS 1 or COS 7, CV1, quail QC1-3 cells, mouse L cells, mouse sarcoma cells, Bowes melanoma cells and Chinese hamster ovary (CHO) cells. Exemplary mammalian host cells in accordance with the present invention are CHO cells. Other suitable eukaryotic host cells include, without being limiting, chicken cells, such as e.g. DT40 cells, or yeasts such as Saccharomyces cerevisiae, Pichia pastoris, Schizosaccharomyces pombe and Kluyveromyces lactis. Insect cells suitable for expression are e.g. Drosophila S2, Drosophila Kc, Spodoptera Sf9 and Sf21 or Trichoplusia Hi5 cells. Suitable zebrafish cell lines include, without being limiting, ZFL, SJD or ZF4.

The described vector(s) can either integrate into the genome of the host or can be maintained extrachromosomally. Once the vector has been incorporated into the appropriate host, the host is maintained under conditions suitable for high level expression of the nucleic acid molecules, and as desired, the collection and purification of the antibody of the invention may follow. Appropriate culture media and conditions for the above described host cells are known in the art.

In one embodiment, the recited host is a mammalian cell, such as a human cell or human cell line. In a further embodiment, the host cell transformed with the vector(s) of the invention is HEK293 or CHO. In yet a further embodiment, the host cell transformed with the vector(s) of the invention is CHO. These host cells as well as suitable media and cell culture conditions have been described in the art, see e.g. Baldi L. et al., Biotechnol Prog. 2005 January-February; 21(1):148-53, Girard P. et al., Cytotechnology 2002 January; 38(1-3):15-21 and Stettler M. et al., Biotechnol. Prog. 2007 November-December; 23(6):1340-6.

In a preferred embodiment, the host cell is a eukaryotic cell. In a particular embodiment, the cell is a HEK cell. In another particular embodiment, the host cell is a CHO cell.

The host cell, in accordance with the present invention, comprises either one vector encoding both the light chain and heavy chain variable regions as defined herein above or it comprises two separate vectors, wherein one vector carries a nucleic acid molecule encoding a light chain variable region in accordance with the present invention and the second vector carries a nucleic acid molecule encoding a matching heavy chain variable region in accordance with the present invention.

When recombinant expression vectors encoding the heavy and/or light chain of the antibody of the invention as disclosed herein are introduced into host cells, the antibodies or antibody antigen binding fragments are produced by culturing the host cells for a period of time sufficient to allow for expression of the antibody or antigen binding fragment in the host cell or, preferably, to allow for secretion of the antibody or antigen binding fragment into the culture medium in which the host cells are grown. Antibodies and/or antigen binding fragments can be recovered from the culture medium using standard protein purification methods. Methods for purification of antibodies are well known in the art. Exemplary purification methods are described in the appended Examples.

Consequently, the invention also provides a method for the production of a monoclonal antibody or antigen-binding fragment specifically binding to Aβ42 as disclosed herein. The method comprises culturing a host cell of the invention under suitable conditions and isolating the antibody produced. By purification steps, e.g. as described in the appended Examples an isolated monoclonal antibody of the invention or antigen-binding fragment thereof can be derived. The invention further provides an antibody or an antigen-binding fragment obtainable by any of the methods disclosed herein.

The transformed host cells can be grown in bioreactors and cultured according to techniques known in the art to achieve optimal cell growth. The antibody and/or antibody antigen binding fragment of the invention can then be isolated from the cell fraction or growth medium by any conventional means such, but not limited to, affinity chromatography (for example using a fusion-tag such as the Strep-tag II or the His₆ tag), gel filtration (size exclusion chromatography), anion exchange chromatography, cation exchange chromatography, hydrophobic interaction chromatography, high pressure liquid chromatography (HPLC), reversed phase HPLC or immunoprecipitation.

It will be appreciated that variations on the above procedures are within the scope of the present invention. For example, recombinant DNA technology may be used to remove or modify the DNA sequences encoding the antibodies and/or antibody antigen binding fragments disclosed herein, e.g. encoding the heavy and/or light chain variable domains as defined herein above. For example, recombinant DNA technology may be used to remove parts of the encoding sequence(s) that are not necessary for maintaining specific and selective binding to the antigen(s) of interest. The molecules expressed from such truncated DNA molecules are also encompassed by the antibodies of the invention. Additionally, also provided are multivalent antibodies or antigen-binding fragments comprising a heavy and/or a light chain variable domain of the invention (e.g. forming and antibody Fv domain that specifically and selectively binds Aβ42) at least twice (preferably four, five, six, seven or eight times). Further, provided are multivalent antibodies or antibody fragments comprising a heavy and/or a light chain or fragment thereof of the invention (e.g. forming and antibody Fab or F(ab)2 domain that specifically and selectively binds Aβ42) at least twice (preferably four, five, six, seven or eight times).

Antibody derivatives can be produced, for example, by adding exogenous sequences to modify immunogenicity or reduce, enhance or modify binding, affinity, on-rate, off-rate, avidity, specificity, half-life, or any other suitable characteristic.

In a fifth aspect, the present invention provided a composition comprising an antibody of the invention, a polynucleotide (or set of polynucleotides) of the invention, a vector of the invention, or a host cell of the invention. In a preferred embodiment, the composition is a diagnostic composition, i.e. a composition for use in diagnostic applications. In preferred embodiments, the composition is for use in an in vitro diagnostic test for detecting or quantifying Aβ42. In a preferred embodiment, the diagnostic composition may be a reagent for an immunoassay for detecting or quantifying Aβ42. The diagnostic composition is preferably configured such that it allows for detection of Aβ42, in a sample obtained from a subject. The sample is preferably a blood sample (e.g. whole blood, serum or plasma). In embodiments, the sample is plasma or serum.

In embodiments, the composition of the invention is a composition for in vitro detection (preferably quantification) of Aβ42 in a sample (e.g. blood sample, such as plasma or serum), preferably using an immunoassay. In embodiments, the immunoassay is a heterogeneous immunoassay. In embodiments, the immunoassay is a sandwich immunoassay.

In a sixth aspect, provided herein is a method for determining the presence and/or level of Aβ42 in a sample. The method comprises: (a) contacting an antibody or antigen binding fragment thereof specifically recognizing Aβ42 according to the first aspect of the invention with the sample under conditions allowing the binding of the antibody or the antigen binding fragment to Aβ42 in the sample; and (b) determine the presence or level of Aβ42 in the sample. Determining the presence or level of Aβ42 may be achieved by detecting the binding of the antibody or the antigen binding fragment of (a) to the Aβ42 comprised in the sample.

The sample as used in the method of the sixth method may be any sample comprising or suspected to comprise Aβ42. In embodiments, the sample may be a body fluid. Non-limiting Examples for body fluid samples are cerebrospinal fluid (CSF) and blood (e.g. whole blood, plasma or serum). In embodiments, the sample is serum or plasma. In embodiments, the sample is plasma.

The subject from which the sample used in the method has been obtained is preferably a human being.

In embodiments, the method according to the sixth aspect comprises a sandwich immunoassay and the monoclonal antibody or antigen-binding fragment according to the first aspect may be used as one binding agent (e.g. as a capture agent or detection agent). As second binding agent in such sandwich assay, an Aβ-specific binding agent specifically binding to any of amino acids 1 to 42 (i.e. Aβ specific but not Aβ42 specific) and not competing with binding to Aβ42 with the antibody or antigen-binding fragment of the first aspect of the invention. may be used (e.g. as detection or capture agent). Exemplary but not limiting Examples for such binding agents are 3D6, 6E10 (BioLegend cat. no. 803004) and 1E8 (Nanotools cat. no. 0315-100/bA4N-1E8). In a preferred embodiment, the second binding agent is 3D6, as defined herein below.

Accordingly, the method of the sixth aspect of the invention may comprise contacting the sample for, during or after (a) with an Aβ binding agent (e.g. an antibody) specifically binding to any of amino acids 1 to 42 (i.e. Aβ specific but not Aβ42 specific) and not competing with binding to Aβ42 with the antibody or antigen-binding fragment of the first aspect of the invention. The method may comprise the formation of a sandwich complex/detection complex between the Aβ42 comprised in the sample, the antibody or antigen-binding fragment of the first aspect of the invention and the Aβ specific binding agent. Determining the presence or level of Aβ42 in the sample may consequently comprise detecting formation of said sandwich complex. In embodiments, the sandwich complex formed may be separated from free binding agents, e.g. via binding to a solid phase (e.g. via a capture label attached to one of the binding agents). In embodiments, the separation of the sandwich complex may comprise binding the sandwich complex to microbeads (e.g. magnetic beads and separating these beads (e.g. via magnetic force) from the remaining components, in particular free detection agent and/or detection label.

In embodiments, the binding of the antibody or antigen-binding fragment according to the first aspect of the invention to Aβ42 may be determined using a detection label. Non-limiting examples for classes and specific detection labels are disclosed elsewhere herein and may applied to this aspect mutatis mutandis. The detection label can be attached to the antibody or antigen binding fragment of the invention. Alternatively, if a second binding agent is used (e.g. an Aβ binding agent, such as an antibody, that specifically binds to any of amino acids 1 to 42 (i.e. Aβ specific but not Aβ42 specific) and does not competing with binding to Aβ42 with the antibody or antigen-binding fragment of the first aspect of the invention), the detection label may be attached thereto.

The method of the sixths aspect of the invention may comprise or be an heterogeneous immunoassay. The antibody or antigen binding fragment of the first aspect of the invention may be used as a capture agent, i.e. an agent that is bound or can be bound to a solid surface (e.g. a micro bead, or a magnetic microbead). Alternatively, the antibody or antigen binding fragment of the first aspect of the invention may be used as a detection agent.

In embodiments, the method comprises a heterogeneous sandwich immunoassay using two Aβ42 binding agents: a capture binding agent and a detection binding agent. The first binding agent may be an Aβ42 specific antibody or an antigen-binding fragment of the first aspect of the invention. The first binding agent may be the capture agent or the detection agent. In embodiments, the first binding agent is the capture agent.

The sample as used in the method according to the sixth aspect of the invention may be a sample previously obtained from a subject (e.g. a blood sample such as plasma or serum), i.e. the method may be an in vitro method. The sample may have been obtained from a subject that is suspected of having or developing amyloid positivity and/or a pathological condition associated therewith, e.g. Alzheimer's disease. In embodiments, the subject may suffer from neurological symptoms and/or have a genetic predisposition (e.g. may be an ApoE4 carrier) for developing Alzheimer's disease. In embodiments, the subject from which the sample has been obtained may be cognitively normal or may show mild cognitive symptoms.

In a seventh aspect, the present invention provides for the use of the antibody or antigen binding fragment specifically binding Aβ42 as provided herein for detecting or quantifying Aβ42 in a sample (e.g. a blood sample such as serum or plasma).

What is disclosed herein elsewhere, especially the embodiments of the method for determining the presence and/or level of Aβ42 in a sample applies mutatis mutandis.

In an eighth aspect, provided herein is a method for aiding in detecting amyloid positivity in a subject. In other words, the method may be a method for diagnosing amyloid positivity in a subject. The method may comprises a) determining the level of Aβ42 in the sample using the antibody or antigen-fragment of the invention and b) comparing the determined level of Aβ42 to a predetermined reference level of Aβ42, wherein an altered (e.g. decreased) level of Aβ42 relative to the reference level is indicative for amyloid positivity. Alternatively or additionally, said method may be a method for aiding in the detection whether a subject has or is at risk of developing an amyloid-positive dementia (e.g. Alzheimer). In these embodiments, a decreased level of Aβ42 is indicative relative to the reference level is indicative for an amyloid-positive dementia or an increased risk of developing it.

The embodiments disclosed herein elsewhere with respect to the method for determining the presence and/or level of Aβ42 apply mutatis mutandis to the methods according to the eighth aspect.

In a particular preferred embodiment, the sample is a blood sample, e.g. serum or plasma.

One aim of the present invention is provide a method for reliably identifying an individual as having (preferably at an early stage) or being at risk of developing an amyloid-positive dementia. Accordingly, the individual may already show signs and symptoms of dementia and the method may be used in order to identify the present dementia as amyloid-positive or amyloid-negative. Alternatively, the individual may not yet show signs and symptoms of dementia. In this case the method may be used to identify the individual as being at risk of developing an amyloid-positive dementia.

Determining the level of Aβ42 in the sample using the antibody or antigen-fragment of the invention may comprise (a contacting the antibody or the antigen binding fragment according to the invention with the sample that as been obtained by the subject under conditions and for a time allowing the binding of the antibody or the antigen binding fragment Aβ42; and determine the level of Aβ42 in the sample by detecting the binding of the antibody or the antigen binding fragment of the invention to Aβ42.

The method may further comprise assessing based on the comparison of the determined Aβ42 level with the reference level whether a subject is amyloid positive, has an amyloid-positive dementia or an increased risk of developing it, and/or is to be selected for subsequent CSF based biomarker analysis or PET diagnostic.

In embodiments, the reference level may be a level as determined in a reference cohort comprising amyloid positive and negative individuals, e.g. as determined by CSF biomarkers (e.g. Elecsys® CSF pTaul81/Aβ42 ratios, for example using a cut-off of 0.024) or PET scan analysis. A reference cohort may have at least 50, preferably at least 100 or at least 500 members.

In embodiments, the reference level may be a level detected in an earlier sample of the same subject, e.g. a baseline Aβ42 level.

In embodiments, the method may comprise a) determining the level of Aβ42 in the sample using the antibody or antigen-fragment of the invention b) determining the level of Aβ40 in the sample; c) determining a combined value (e.g. a ratio, such as an Aβ42/Aβ40 ratio) from the level of Aβ42 and the level of Aβ40 and d) comparing the combined value to a predetermined reference combined value, wherein an altered combined value is indicative for amyloid positivity. A skilled person will appreciate that a combined value can be generated in different ways so that depending on the configuration either an increased or decreased combined value compared to the reference combined value indicates amyloid positivity. If Aβ42/Aβ40 ratio is used a decreased combined value is indicative for amyloid positivity.

Accordingly, provided herein is a method for aiding in detecting amyloid positivity in a subject, said method comprising: a) determining the level of Aβ42 in the sample using the antibody or antigen-fragment specifically binding to Aβ42 of the invention b) determining the level of Aβ40 in the sample; c) determining the Aβ42/Aβ40 ratio from the level of Aβ42 and the level of Aβ40 (i.e. the ratio between the determined level of Aβ42 and the determined level of Aβ40) and d) comparing the combined value to a predetermined reference combined value, wherein an altered (preferably decreased) combined value is indicative for amyloid positivity.

As demonstrated in the appended Example, determining the Aβ42/Aβ40 ratio comprising the quantification of Aβ42 using an immunoassay using the Aβ42 antibody of the invention significantly improved the clinical performance in identifying amyloid positivity.

In embodiments, the reference combined value may be a combined value as determined in a reference cohort comprising amyloid positive and negative individuals, e.g. as determined by CSF biomarkers (e.g. Elecsys® CSF pTaul81/Aβ42 ratios, for example using a cut-off of 0.024) or PET scan analysis. A reference cohort may have at least 50, preferably at least 100 or at least 500 members. In embodiments, the reference cohort may be as the cohort employed in Example 4.

In embodiments, the reference combined value may be a combined value detected in an earlier sample of the same subject, e.g. a baseline combined value.

There exists a variety of methods for measuring Aβ40 levels. An assay for Aβ40 specifically measures Aβ40, but not e.g., Aβ42 or Aβ43. The measurement of Aβ40 is typically based on binding specific for the COOH-terminus of the 1-40 Aβ sequence. Commercially available products for measuring Aβ40 include Amyloid beta 40 Human ELISA Kit (Thermo Fisher Scientific, Waltham, MA, USA), Simoa™ Aβ40 immunoassay (Quanterix Corporation, Lexington, MA, USA), and Amyloid β1-40 Assay Kit (Cisbo Assays, Codolet, France). Preferably, Aβ40 is measured using the Elecsys® ECL technology on in an Elecsys® measuring cell according to the manufacturer's instructions and as detailed in the Examples.

A “combined value”, or also referred to as combined score, in the context of the present invention refers to a value obtained by a mathematical procedure combining the level of Aβ42 and a second biomarker (e.g. Aβ40 level) obtained from the same sample. The combined value is calculated according to a mathematical operation, preferably an arithmetic operation using the amount/concentration of the respective biomarkers. The result of the operation is a value, namely the combined value. The combined value may then be compared to the combined value of the control, which has been obtained using the same mathematical procedure. In the invention usually either the amount or the concentration, preferably the concentration, of all markers is used in order to obtain the combined value for the individual and the control. Preferably, the combined value is obtained by adding the values obtained for the concentrations of the markers. In another preferred embodiment, the combined value is obtained by weighted calculation of the amount or concentration of the marker molecules in the samples. This means that the markers are given different weightings in the mathematical procedure.

For example, a logistic regression analysis may be performed with a binary outcome “amyloid-positive” and “amyloid-negative” as the dependent variable and the combination of the biomarker levels as the independent variables. Classification accuracy may be assessed by Area under the ROC (Receiver-Operating Characteristic) curve (AUC) and sensitivity and specificity may be calculated (see below).

In embodiments, the accuracy for determining amyloid positivity in samples of a reference population using the method determining an Aβ42/Aβ40 ratio (or vice versa) is higher than for the same method using Aβ42 antibodies 21F12 and/or H31L21, respectively. The accuracy is preferably determined by a ROC analysis and the AUC (area under the curve) for detecting amyloid positivity in the samples of the reference population (with known amyloid status) is used as measure for accuracy. In embodiments, the AUC of the Aβ42/Aβ40 ratio using an Aβ42 assay of the invention for detecting amyloid positivity in the samples of the reference population may be at least 0.8, preferably at least 0.85 and most preferably at least 0.86.

In embodiments the monoclonal antibody or antigen-binding fragment of the invention may be a monoclonal antibody or an antigen-binding fragment thereof specifically binding to Aβ42, wherein when the antibody is used in a sandwich immunoassay for Aβ42 in blood samples (e.g. serum or plasma samples) the accuracy for determining amyloid positivity in samples of a reference population using an Aβ42/Aβ40 ratio (or vice versa) is at least as good as for the same sandwich immunoassay using the antibody of the invention having a heavy chain of SEQ ID NO:9 and a light chain of SEQ ID NO:10, respectively. The accuracy is preferably determined by a ROC analysis and the AUC (area under the curve) for detecting amyloid positivity in the samples of the reference population (with known amyloid status) is used as measure for accuracy.

The reference population may, for example be a reference cohort as used in the appended Examples, e.g. may comprise amyloid positive and amyloid negative subjects. The amyloid positive subjects may have no, mild cognitive impairment or cognitive impairment.

The reference read-out for amyloid positivity used for the ROC analysis may be (e.g. Elecsys® CSF pTaul81/Aβ42 ratios as measured in CSF samples, for example using a cut-off of 0.024) or PET scan analysis. In preferred embodiments, the reference read-out for amyloid positivity used for the ROC analysis of the samples of the reference population is Elecsys® CSF pTaul81/Aβ42 ratio as measured in CSF samples, preferably using a cut-off of 0.024.

In a ninth aspect, the present invention provides for kit comprising the antibody or antigen binding fragment specifically binding to Aβ42 of the invention. The kit may in particular be a kit for detecting and/or quantifying Aβ42 in a sample. The sample may be any sample as defined herein elsewhere.

All embodiments and combinations thereof disclosed herein with respect to the antibody or antigen binding fragment specifically binding to Aβ42 of the invention apply mutatis mutandis.

In embodiments, the kit provided herein may be a kit for a sandwich immunoassay for specifically detecting and/or quantifying Aβ42.

Accordingly, in embodiments, the kit may further comprise a second binding agent (e.g. an antibody) that specifically binds to amino acids 1 to 42 of Aβ but does not compete for binding to Aβ42 with the antibody or antigen binding fragment of the invention. Competition of two antibodies can be assessed with standard methods such as ELISA techniques, Elecsys® based immunoassay experiments or surface plasmon resonance spectroscopy (e.g. Biacore) based competition experiments. Preferred are surface plasmon resonance spectroscopy competition experiments. An Aβ binding agent that does not compete for binding with the antibody or antigen-binding fragment of the invention does not reduce the binding of the antibody or antigen-binding fragment of the invention to Aβ42 or an Aβ42 mimicking peptide. A skilled person will appreciate that depending on the measurement method used there will be a certain measurement error. For instance, an antibody is considered to not compete with the antibody or antigen-binding fragment of the invention, if the binding of the antibody or antigen-binding fragment of the invention to Aβ42 or the Aβ42 mimicking peptide is diminished by 10% or less, preferably 5% or less.

The second binding agent may in principle be a binding agent of any type, e.g. an antibody, an aptamer or any other specific binding partner for amino acids 1-42 of Aβ42 that does not compete for binding to Aβ42 with the antibody or antigen-binding fragment of the invention.

In embodiments, the second binding agent is an antibody specifically binding to amino acids 1-42 of Aβ. Since amino acids 1-42 or large parts thereof are also present in other Aβ peptides, the second binding agent is not specific for Aβ42, but Aβ peptides in general.

Antibodies specifically binding to amino acids 1-42 of Aβ but not competing with compete for binding to Aβ42 with the antibody or antigen-binding fragment of the invention are known in the art. Non-limiting examples for such antibodies are: 3D6, 6E10 (BioLegend cat. no. 803004) and 1E8 (Nanotools cat. no. 0315-100/bA4N-1E8).

In embodiments, the second binding agent may be the antibody 3D6 as defined herein elsewhere.

In embodiments, the second binding agent may specifically bind to an amino acids 1 to 5 of Aβ (i.e. amino acids 672-676 of APP; see database UniProtKB accession number: P05067; Entry version 298). This epitope is recognized by 3D6 (Feinberg H, Alzheimer's Research & Therapy volume 6, Article number: 31, 2014), which is accordingly an exemplary antibody binding to this epitope.

In embodiments, the antibody or antigen-binding fragment of the invention may have a detection label (as defined herein elsewhere) attached thereto. In embodiments, the antibody or antigen-binding fragment of the invention may have a capture label (as defined herein elsewhere) attached thereto.

In embodiments, the kit may comprise the antibody or antigen-binding fragment of the invention having a capture label attached thereto and a second binding agent as defined herein above that has a detection label attached thereto. The capture and detection label may also be attached vice versa.

In embodiments, the kit further comprises microbeads, in particular magnetic microbeads. Said microbeads are preferably configured such that their surfaces can bind to the capture label (e.g. they may be coated with a binding partner to the capture label; e.g. coating with streptavidin or a derivative thereof that binds to the capture label biotin or a derivative thereof). Alternatively, one of the binding agents in the kit may be directly attached to the surface of the microbeads.

In embodiments, the kit of the invention is for conducting any of the methods disclosed herein. What has been said with respect to these methods herein applies mutatis mutandis.

In embodiments, the kit comprises any manufacture, such as instructions for use or the like.

The components of the kit may be comprised in a single container or may be distributed to two or more containers.

The present invention also relates to the following items:
1. A monoclonal antibody or an antigen-binding fragment thereof specifically binding to Aβ42, comprising:
   (i) a heavy chain variable domain (VH) comprising the amino acid sequence of SEQ ID NO: 1 or an amino acid sequence having a sequence identity of at least 70%, preferably 80%, even more preferably 90%, even more preferably 95% and most preferably 99% with SEQ ID NO: 1; and
   (ii) a light chain variable domain (VL) comprising the amino acid sequence of SEQ ID NO: 2 or an amino acid sequence having a sequence identity of at least 70%, preferably 80%, even more preferably 90%, even more preferably 95% and most preferably 99% with SEQ ID NO: 2.
2. The monoclonal antibody or antigen-binding fragment of item 1, wherein the heavy chain variable domain (VH) comprises (a) a CDR-H1 comprising the amino acid sequence of SEQ ID NO: 3 or a variant thereof with one amino acid substitution, (b) a CDR-H2 comprising the amino acid sequence of SEQ ID NO: 4 or a variant thereof with one amino acid substitution, and (c) a CDR-H3 comprising the amino acid sequence of SEQ ID NO: 5 or a variant thereof with one amino acid substitution, and wherein the light chain variable domain (VL) comprises (d) a CDR-L1 comprising the amino acid sequence of SEQ ID NO: 6 or a variant thereof with one amino acid substitution, (e) a CDR-L2 comprising the amino acid sequence of SEQ ID NO: 7 or a variant thereof with one amino acid substitution, and (f) a CDR-L3 comprising the amino acid sequence of SEQ ID NO: 8 or a variant thereof with one amino acid substitution.
3. The monoclonal antibody or antigen-binding fragment of item 1, wherein the heavy chain variable domain (VH) comprises (a) a CDR-H1 comprising the amino acid sequence of SEQ ID NO: 3, (b) a CDR-H2 comprising the amino acid sequence of SEQ ID NO: 4, and (c) a CDR-H3 comprising the amino acid sequence of SEQ ID NO: 5, and wherein the light chain variable domain (VL) comprises (d) a CDR-L1 comprising the amino acid sequence of SEQ ID NO: 6, (e) a CDR-L2 comprising the amino acid sequence of SEQ ID NO: 7, and (f) a CDR-L3 comprising the amino acid sequence of SEQ ID NO: 8.
4. A monoclonal antibody or an antigen-binding fragment thereof specifically binding to Aβ42, comprising a heavy chain variable domain (VH) comprising (a) a CDR-H1 comprising the amino acid sequence of SEQ ID NO: 3 or a variant thereof with one amino acid substitution, (b) a CDR-H2 comprising the amino acid sequence of SEQ ID NO: 4 or a variant thereof with one amino acid substitution, and (c) a CDR-H3 comprising the amino acid sequence of SEQ ID NO: 5 or a variant thereof with one amino acid substitution, and wherein the light chain variable domain (VL) comprises (d) a CDR-L1 comprising the amino acid sequence of SEQ ID NO: 6 or a variant thereof with one amino acid substitution, (e) a CDR-L2 comprising the amino acid sequence of SEQ ID NO: 7 or a variant thereof with one amino acid substitution, and (f) a CDR-L3 comprising the amino acid sequence of SEQ ID NO: 8 or a variant thereof with one amino acid substitution.
5. The monoclonal antibody or antigen-binding fragment of item 4 specifically binding to Aβ42, wherein the heavy chain variable domain (VH) comprises (a) CDR-H1 comprising the amino acid sequence of SEQ ID NO: 3, (b) CDR-H2 comprising the amino acid sequence of SEQ ID NO: 4, and (c) CDR-H3 comprising the amino acid sequence of SEQ ID NO: 5, and a light chain variable domain (VL) comprising (d) CDR-L1 comprising the amino acid sequence of SEQ ID NO: 6, (e) CDR-L2 comprising the amino acid sequence of SEQ ID NO: 7, and (f) CDR-L3 comprising the amino acid sequence of SEQ ID NO: 8.
6. The monoclonal antibody or the antigen-binding fragment of item 4 or 5, wherein
   (i) the heavy chain variable domain (VH) comprises the amino acid sequence of SEQ ID NO: 1 or an amino acid sequence having a sequence identity of at least 70%, preferably 80%, even more preferably 90%, even more preferably 95% and most preferably 99% with SEQ ID NO: 1; and
   (ii) the light chain variable domain (VL) comprises the amino acid sequence of SEQ ID NO: 2 or an amino acid sequence having a sequence identity of at least 70%, preferably 80%, even more preferably 90%, even more preferably 95% and most preferably 99% with SEQ ID NO: 2.
7. The monoclonal antibody or the antigen-binding fragment of any one of items 1 to 6, wherein the heavy chain comprises or consists of the amino acid sequence of SEQ ID NO: 9 or an amino acid sequence having a sequence identity of at least 70%, preferably 80%, even more preferably 90%, even more preferably 95% and most preferably 99% with SEQ ID NO: 9.
8. The monoclonal antibody or the antigen-binding fragment of any one of items 1 to 7, wherein the light chain comprises the amino acid sequence of SEQ ID NO: 10 or an amino acid sequence having a sequence identity of at least 70%, preferably 80%, even more preferably 90%, even more preferably 95% and most preferably 99% with SEQ ID NO: 10.
9. The monoclonal antibody or the antigen-binding fragment of any one of items 1 to 8, wherein the monoclonal antibody or the antigen-binding fragment discriminate Aβ42 from all other Aβ peptides, wherein in embodiments the other Aβ peptides consist of Aβ38, Aβ40 and Aβ43.
10. The monoclonal antibody or the antigen-binding fragment of any one of items 1 to 9, wherein the binding of the monoclonal antibody or the antigen-binding fragment to Aβ42 depends on the C-terminus of the Aβ42 peptide.
11. The monoclonal antibody or the antigen-binding fragment of any one of items 1 to 10, wherein the monoclonal antibody or antigen-binding fragment binds Aβ42 at 37° C. with a dissociation equilibrium constant K_{D} of 20 nM or lower, preferably of 10 nM or lower and most preferably of 7 nM or lower.
12. The monoclonal antibody or the antigen-binding fragment of any one of items 1 to 11, wherein the monoclonal antibody or antigen-binding fragment binds Aβ42 at 37° C. with an association rate constant kₐ of 1*10⁴ M⁻¹s⁻¹ or higher, preferably 1*10⁵ M⁻¹s⁻¹ or higher, even more preferably 1*10⁶ M⁻¹s⁻¹ or higher, even more preferably 3*10⁶ M⁻¹s⁻¹ or higher and most preferably 3.8*10⁶ M⁻¹s⁻¹ or higher.
13. The monoclonal antibody or the antigen-binding fragment of any one of items 1 to 12, wherein the monoclonal antibody or antigen-binding fragment binds Aβ42 at 37° C. with a dissociation rate constant k_{d} of 10⁻² s⁻¹ or lower, preferably 2*10⁻² s⁻¹ or lower and most preferably 2.5 10⁻² s⁻¹ or lower. A low dissociation rate constant ensures high complex stability with Aβ42.
14. The monoclonal antibody or the antigen-binding fragment of any one of items 1 to 13, wherein when the monoclonal antibody or the antigen-binding fragment is used as an Aβ42 specific binding agent in a sandwich immunoassay for detecting and/or quantifying Aβ42 in a blood sample (e.g. serum or plasma sample), the accuracy for determining amyloid positivity in blood samples of a reference population with known amyloid status using an Aβ42/Aβ40 ratio (or vice versa) is higher than for the same Aβ42 sandwich immunoassay using Aβ42 antibodies 21F12 and/or H31L21.
15. The monoclonal antibody or the antigen-binding fragment of any one of items 1 to 14, wherein when the monoclonal antibody or the antigen-binding fragment is used as an Aβ42 specific binding agent in a sandwich immunoassay for detecting and/or quantifying Aβ42 in a blood sample (e.g. serum or plasma sample), the accuracy for determining amyloid positivity in blood samples of a reference population with predetermined amyloid status using an Aβ42/Aβ40 ratio (or vice versa) is at least as good as for the same sandwich immunoassay using an antibody consisting of the heavy chain of SEQ ID NO:9 and the light chain of SEQ ID NO:10.
16. The monoclonal antibody or the antigen-binding fragment of item 14 or 15, wherein the accuracy is assessed by the AUC of a ROC analysis.
17. The monoclonal antibody or the antigen-binding fragment of any one of items 1 to 16, wherein when the monoclonal antibody or the antigen-binding fragment is used as an Aβ42 specific binding agent in an immunoassay (e.g. sandwich immunoassay) for detecting and/or quantifying Aβ42 in a blood sample (e.g. serum or plasma sample), does not show rheumatoid factor (Rf) interference, wherein the blood sample shows Rf interference with the Aβ42 antibodies 21F12 and/or H31L21 when used in an immunoassay.
18. The monoclonal antibody or the antigen-binding fragment of any one of items 1 to 17, when used as Aβ42 specific binding agent in an immunoassay (e.g. sandwich immunoassay) detects the amount of Aβ42 in a blood sample more accurately than other previously described Aβ42 antibodies (e.g. more accurately than 21F12 and/or H31L21) when used in an otherwise identical immunoassay, wherein the blood sample shows Rf interference with other Aβ42 specific binding agents (e.g. the Aβ42 antibodies 21F12 and/or H31L21) and comprise a detectable and predetermined Aβ42 amount.
19. The monoclonal antibody or the antigen-binding fragment of item 17 or 18, wherein the blood sample has an RF activity between 36 IU/ml and 1200 IU/ml, in embodiments between 100 IU/ml and 1200 IU/ml, and in embodiments between 300 IU/ml and 1200 IU/ml.
20. The monoclonal antibody or the antigen-binding fragment of any one of items 17 to 19, wherein the blood sample is spiked with a purified Rf concentrate obtained from a pool of plasma or serum samples that have been obtained from rheumatoid arthritis patients.
21. The monoclonal antibody or the antigen-binding fragment of any one of items 17 to 20, wherein the blood sample is serum or plasma.
22. The monoclonal antibody or the antigen-binding fragment of any one of items 1 to 21, wherein the antibody or antigen-binding fragment does not bind to one or more antibodies of subtype IgM when incubated with a blood sample having an Rf activity of 300 IU/ml or more (e.g. 300 IU/ml to 1200 IU/ml), said blood sample comprising antibodies of subtype IgM binding to an F(ab′)2 region comprising the variable region of the Aβ1-42 antibody 21F12 and/or to an F(ab′)2 region comprising the variable region of the Aβ1-42 antibody H31L21.
23. The monoclonal antibody or antigen binding fragment of any one of items 1 to 22, wherein the heavy chain variable domain comprises the sequence of SEQ ID NO: 1 and the light chain variable domain comprises the sequence of SEQ ID NO: 2.
24. The monoclonal antibody or antigen binding fragment of any one of items 1 to 22, wherein the heavy chain variable domain comprises the sequence of SEQ ID NO: 9 and the light chain variable domain comprises the sequence of SEQ ID NO: 10.
25. The monoclonal antibody or antigen binding fragment of any one of items 1 to 24, wherein the antibody or antigen binding fragment is labeled with a capture label.
26. The monoclonal antibody or antigen binding fragment of any one of items 1 to 25, wherein the antibody or antigen binding fragment is labeled with a detection label.
27. A polynucleotide or a set of polynucleotides encoding
   (i) the heavy chain or heavy chain variable domain of the monoclonal antibody or antigen binding fragment according to any one of items 1 to 26 and 58 and 59, and/or
   (ii) the light chain or light chain variable domain of the monoclonal antibody or antigen binding fragment according to any one of items 1 to 26 and 58 and 59.
28. A vector comprising the polynucleotide or set of polynucleotides according to item 27.
29. A host cell comprising the polynucleotide or set of polynucleotides according to item 27, or the vector according to item 28.
30. The host cell of item 29, wherein the host cell is a prokaryotic cell or an eukaryotic cell.
31. The host cell of item 29 or 30, wherein the host cell is a HEK or a CHO cell.
32. A method of producing the monoclonal antibody or the antigen-binding fragment of any one of items 1 to 26 and 58 and 59 comprising culturing the host cell according to any one of items 29 to 31 and isolating said antibody.
33. An antibody or antigen-binding fragment according to any one of items 1 to 26 obtainable by the method of item 32.
34. A composition comprising the antibody or antigen-binding fragment according to any one of items 1 to 26 and 58 and 59, the polynucleotide according to item 27, the vector according to item 28, or the host cell according to any one of items 29 to 31.
35. A composition comprising the antibody or antigen-binding fragment according to any one of items 1 to 26 and 58 and 59 that is a diagnostic composition.
36. Use of the antibody or antigen binding fragment of any one of items 1 to 26 and 58 and 59 as an Aβ42 binding agent for detecting or quantifying Aβ42 in a sample.
37. The use of item 36, wherein the sample is a blood sample.
38. The use of item 36 or 37, wherein the sample is serum or plasma.
39. The use of any one of items 36 to 38, wherein the detection or quantification involves an immunoassay, in embodiments a sandwich immunoassay.
40. The use of any one of items 36 to 39, wherein the Aβ42 in the sample is detected without Rf interference.
41. The use of any one of item 36 to 39, wherein the sample shows Rf interference with the F(ab′)₂ region of the Aβ1-42 antibody 21F12 and/or with the F(ab′)₂ region of the Aβ1-42 antibody H31L21.
42. An in vitro method for determining the presence and/or the level of Aβ42 in a sample, said method comprising:
   (a) contacting the antibody or the antigen binding fragment according to any one of items 1 to 26 and 58 and 59 with the sample under conditions allowing the binding of the antibody or the antigen binding fragment of any one of items 1 to 26 and 58 and 59 to Aβ42 in the sample; and
   (b) determine the presence or level of Aβ42 in the sample by detecting the binding of the antibody or the antigen binding fragment according to any one of items 1 to 26 and 58 and 59 to Aβ42.
43. An in vitro method for aiding in the detection of amyloid positivity in a subject, said method comprising
   (a) determine the level of Aβ42 using the antibody or the antigen binding fragment according to any one of items 1 to 26 and 58 and 59; and
   (b) comparing the determined level of Aβ42 to a predetermined reference level of Aβ42, wherein an altered (e.g. decreased) level of Aβ42 relative to the reference level is indicative for amyloid positivity.
44. An in vitro method for aiding in the detection of amyloid positivity in a subject, said method comprising
   a) determining the level of Aβ42 in the sample using the antibody or antigen-fragment according to any one of items 1 to 26 and 58 and 59
   b) determining the level of Aβ40 in the sample;
   c) determining a combined value (e.g. an Aβ42/Aβ40 level ratio) from the level of Aβ42 and the level of Aβ40; and
   d) comparing the combined value to a predetermined reference combined value, wherein an altered (e.g. decreased) combined value is indicative for amyloid positivity.
45. The method of item 43 or 44, wherein the method further comprises assessing based on the comparison in whether a subject is amyloid positive.
46. The method of any one of items 43 to 45, wherein the subject is cognitively unimpaired or cognitively impaired.
47. The method of any one of items 42 to 46, wherein the sample is a body fluid, in embodiments a blood sample.
48. The method of any one of items 42 to 47, wherein the sample is a serum or plasma sample.
49. The method of any one of items 42 to 48, wherein the level of Aβ42 is determined with a sandwich immunoassay using the antibody or antigen-binding fragment of any one of items 1 to 26 and 58 and 59 as a first Aβ42 binding agent and using a Aβ specific binding agent binding to Aβ42 but not competing for binding to Aβ42 with the antibody or antigen-binding fragment of any one of items 1 to 26 and 58 and 59 is used as a second Aβ42 binding agent.
50. The method of any one of items 42 to 49, wherein the level of Aβ42 is determined with a sandwich immunoassay using the antibody or antigen-binding fragment of any one of items 1 to 26 and 58 and 59 as a capture agent, wherein the sample is contacted with a Aβ specific detection agent that binds to Aβ42 but does not compete for binding to Aβ42 with the antibody or antigen-binding fragment of any one of items 1 to 26 and 58 and 59, wherein a detection complex comprising Aβ42 from the sample, the antibody or antigen-binding fragment of any one of items 1 to 26 and 58 and 59 and the detection agent is formed, and wherein the detection of the Aβ42 level in the sample comprises detecting the level of the detection complex.
51. The method of item 50, wherein the method comprises separating the detection complex from unbound detection complex.
52. The method of item 50 or 51, wherein the capture compound is bound to a solid surface, in embodiments magnetic particles, in embodiments magnetic microbeads.
53. A kit for detecting and/or quantifying Aβ42 in a sample, said kit comprising the antibody or antigen binding fragment of any one of items 1 to 26 and 58 and 59.
54. The kit of item 53, wherein the kit further comprises a second binding agent specifically binding to Aβ, wherein the second binding agent binds to Aβ42 but does not compete for binding to Aβ42 with the antibody or antigen-binding fragment of any one of items 1 to 26 and 58 and 59.
55. The kit of item 53 or 54, wherein the second binding agent specifically binds to Aβ42 in the region between AS 1 and 5 thereof (i.e. the N-terminal 5 amino acids of Aβ42)
56. The kit of any one of items 53 to 55, wherein the second binding agent is the Aβ antibody 3D6.
57. The kit of any one of items 53 to 56, wherein the kit further comprises magnetic particles, in embodiments magnetic microbeads.
58. The monoclonal antibody or antigen-binding fragment of any one of items 1 to 26, wherein the VH comprises the amino acids at positions 12, 19, 20, 23, 24, 29, 37, 72, 77, 79, 82, 95, 97, 113, 114 and 118 of SEQ ID NO: 1 at the positions corresponding thereto.
59. The monoclonal antibody or antigen-binding fragment of any one of items 1 to 26, wherein the VL comprises the amino acids at positions 10, 12, 14, 15, 17, 18, 19, 41, 42, 44, 51, 74, 92, 105, 110, 111 and 112 of SEQ ID NO: 2 at the positions corresponding thereto.

As evident from the above, the present invention provides antibodies or antigen-binding fragments that specifically bind to Aβ42 and related aspects as defined herein above.

Amyloid beta (Aβ, Ab or Abeta) as used herein denotes peptides of 36-43 amino acids that are the main component of the amyloid plaques found in the brains of people with Alzheimer's disease (Hamley I W, 2012; Chemical Reviews. 112 (10): 5147-92). The peptides derive from the amyloid precursor protein (APP; see database UniProtKB accession number: P05067; Entry version 298), which is cleaved by beta secretase and gamma secretase to yield Aβ in a cholesterol dependent process (Wang H et al., Proceedings of the National Academy of Sciences of the United States of America. 118 (33): e2102191118). Aβ molecules can aggregate to form flexible soluble oligomers which may exist in several forms. It is now believed that certain misfolded oligomers (known as “seeds”) can induce other Aβ molecules to also take the misfolded oligomeric form, leading to a chain reaction akin to a prion infection. The oligomers are toxic to nerve cells (Haas C, Nature Reviews. Molecular Cell Biology. 8 (2): 101-12).

The two most abundant alloforms of amyloid peptides (found in amyloid deposits in the brain) are 40 and 42 amino acids long (designated Aβ40 and Aβ42, respectively). Despite the small structural difference between these two peptides, they display distinct clinical, biological, and biophysical behavior. Human Aβ40 and Aβ42 consist of amino acids 672-711 and 672-713, respectively, of protein APP (see database UniProtKB accession number: P05067; Entry version 298).

Herein referred is to the following Aβ peptides: Aβ38, Aβ40, Aβ42 and Aβ43.

Aβ38 (also referred to as Ab38, Abeta38, Aβ1-38 or Abeta1-38) corresponds to amino acids 672-709 of APP (see database UniProtKB accession number: P05067; Entry version 298). Aβ40 (also referred to as Ab40, Abeta40, Aβ1-40 or Abeta1-40) corresponds to amino acids 672-711 of APP (see database UniProtKB accession number: P05067; Entry version 298). Aβ42 (also referred to as Ab42, Abeta42, Aβ1-42 or Abeta1-42) corresponds to amino acids 672-713 of APP (see database UniProtKB accession number: P05067; Entry version 298). Aβ43 (also referred to as Ab43, Abeta43, Aβ1-43 or Abeta1-43) corresponds to amino acids 672-714 of APP (see database UniProtKB accession number: P05067; Entry version 298).

Alzheimer's disease has been identified as a protein misfolding disease (proteopathy), most likely caused by plaques of abnormally folded amyloid β protein and tangles of tau protein in the brain. Plaques are made up of small peptides, 36-43 amino acids in length, called amyloid β peptides (Aβ). Aβ refers to a group of fragments from the larger amyloid β precursor protein (APP), a transmembrane protein that penetrates through the neuron's membrane. APP is critical to neuron growth, survival, and post-injury repair. In Alzheimer's disease, gamma secretase and beta secretase act together in a proteolytic process which causes APP to be divided into smaller fragments. One of these fragments gives rise to fibrils of amyloid β, which then form clumps that deposit outside neurons in dense formations known as senile plaques. Tangles are caused by abnormal aggregation of the tau protein, which usually stabilizes microtubules of the cytoskeleton when phosphorylated. In Alzheimer's disease, Tau becomes hyperphosphorylated and begins to pair with other threads, creating neurofibrillary tangles and disintegrating the neuron's transport system.

Exactly how disturbances of production and aggregation of the amyloid β peptide give rise to the pathology of Alzheimer's disease is not known. The amyloid hypothesis traditionally points to the accumulation of amyloid β peptides as the central event triggering neuron degeneration. Accumulation of aggregated amyloid fibrils, which are believed to be the toxic form of the protein responsible for disrupting the cell's calcium ion homeostasis, induces programmed cell death (apoptosis). It is also known that Aβ selectively builds up in the mitochondria in the cells of Alzheimer's-affected brains, and it also inhibits certain enzyme functions and the utilization of glucose by neurons.

Pathological changes in the β-amyloid metabolism are among the earliest alterations during AD development known so far that can be utilized diagnostically. They are, for example, reflected by the decrease in the CSF or plasma concentrations of Aβ42 or by the decrease of the Aβ42/Aβ40 ratio. The mechanism for the decreased concentration is not fully understood but one possible explanation is that due to sequestration of Aβ42 in brain plaques, less can be cleared into the CSF or blood (Blenow K and Zetterberg H, Journal of Internal Medicine, Vol 284, issue 6, p. 643 to 663, 2018; and Lewczuk P et al., Adv Med Sci, 2015 March; 60(1):76-82. doi: 10.1016/j.advms.2014.11.002). The Aβ42/Aβ40 ratio may show superior diagnostic performance for detection of AD over Aβ42 alone since it normalizes against different baseline levels of Aβ peptides that may occur between different individuals (Wiltfang J, Journal of Neurochemistry, Vol 101, Issue 4, p. 1053-1059, 2007).

The terms “amyloid positivity” or “amyloid positive” as used herein mean that the subject is positive for Aβ plaques according to amyloid PET, CSF assay (e.g. Elecsys® pTau/Aβ42) or any other gold standard technology for detecting Aβ plaques.

The terms “antibody”, “antibodies”, and analogous terms as used herein relate to full immunoglobulin molecules and encompass naturally-occurring forms of antibodies (including but not limited to IgG, IgA, IgM, IgE) as well as recombinant antibody constructs including but not limited to single-chain antibodies, chimeric antibodies, humanized antibodies, antibody-fusion proteins, and multi-specific antibodies; as well as antigen binding fragments and derivatives of all of the foregoing. The terms “antibody”, “antibodies”, and analogous terms as used herein also refer to antigen binding fragments thereof, which may be referenced herein as antibody antigen binding fragment, and/or, simply antigen binding fragment. These terms refer to one or more fragments of an antibody that retain the ability to specifically bind to the target antigen, i.e. specifically bind to Aβ42, as known in the art including but not limited to antigen binding fragments comprising an Fv domain, i.e., paired heavy and light chain variable domains, such as Fab, Fab′, F(ab′)₂, and Fv fragments as well as recombinant constructs such as single-chain Fv domains, known in the art as scFvs. The terms also includes antibody antigen binding fragments that comprise a single, unpaired heavy or light chain variable domain as known in the art that retains the ability to specifically and selectively bind antigen as defined herein, including but not limited to single domain antibodies (also referenced in the art as sdAbs, dAbs, and/or nanobodies) and VHH domains based on the heavy chains of camelids. The term antibody also includes multivalent antibodies or antigen-binding fragments comprising a heavy and/or a light chain variable domain of the invention (e.g. forming and antibody Fv domain that specifically and selectively binds Aβ42) at least twice (preferably four, five, six, seven or eight times). Further, included are multivalent antibodies or antibody fragments comprising a heavy and/or a light chain or fragment thereof of the invention (e.g. forming and antibody Fab or F(ab)2 domain that specifically and selectively binds Aβ42) at least twice (preferably four, five, six, seven, eight or ten times). Exemplary multivalent formats included by the term antibody herein are disclosed in WO2019/057816, which is incorporated herein in by reference in its entirety.

In certain embodiments the monoclonal antibody of the invention may be a full-immunoglobulin, Fab, Fab′, F(ab′)₂, Fv or scFv. In a specific embodiment, the monoclonal antibody of the invention may be a F(ab′)₂ fragment or an Fab′ fragment.

A “Fab fragment” as used herein is comprised of one light chain and the C_{H}1 and variable regions of one heavy chain. The heavy chain of a Fab molecule cannot form a disulfide bond with another heavy chain molecule.

A “Fab′ fragment” as used herein contains one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv” comprises the variable regions from both the heavy and light chains, but lacks the constant regions. “Single-chain Fvs” (also abbreviated as “scFv”) are antibody fragments that have, in the context of the present invention, the V_{H} and V_{L} domains of an antibody, wherein these domains are present in a single polypeptide chain. Generally, the scFv polypeptide further comprises a polypeptide linker between the V_{H} and V_{L} domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. 113 (1994), 269-315.

Antibodies may be polyclonal or monoclonal. The antibodies of the invention are monoclonal. The term “monoclonal as used herein with reference to an antibody or antigen binding fragment thereof, refer to a population of antibody polypeptides or fragments thereof produced from a single B cell clone, which population contains only one species of an antigen binding site capable of immunoreacting with a particular epitope of an antigen. This is in contrast with “polyclonal” antibodies and compositions, which term(s) refer to a population of antibody polypeptides or antigen binding fragments that contain multiple species of antigen binding sites. Also included are modified forms of monoclonal antibodies of the invention such as humanized or chimeric versions thereof, as well as recombinant antibody constructs, such as antibody (or antigen binding fragment)-fusion proteins, wherein the antibody or antigen binding fragment comprises (an) additional domain(s), e.g. for the isolation and/or preparation of recombinantly produced antibody/fragment/constructs. For example, the monoclonal antibodies to be used in accordance with the present disclosure may be made by a variety of techniques, including, for example, the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Haemmerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, PNAS USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., PNAS USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol.13: 65-93 (1995).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g., U.S. Pat. No. 4,816,567 and Morrison et al., PNAS USA 81:6851-6855 (1984)). Chimeric antibodies include PRIMATIZED® antibodies wherein the antigen-binding region of the antibody is derived from an antibody produced by, e.g., immunizing macaque monkeys with the antigen of interest.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “substitution”, “exchange” or “mutate” as used herein in the context of amino acids refers to the replacement of an amino acid with another amino acid. The deletion of an amino acid at a certain position and the introduction of one (or more) amino acid(s) at a different position is explicitly not encompassed by the term “substitution”. As noted, the present invention encompasses conservative or highly conservative amino acid substitutions as have been defined herein above.

The term “insertion”, in accordance with the present invention, refers to the addition of one or more amino acids to the specifically recited amino acid sequence, wherein the addition is not to the N- or C-terminal end of the polypeptide.

The term “addition”, in accordance with the present invention, refers to the addition of one or more amino acids to the specifically recited amino acid sequence, either to the N- or C-terminal end of the polypeptide, or to both.

The term “deletion”, as used in accordance with the present invention, refers to the loss of one or more amino acids from the specifically recited amino acid sequence.

Amino acids are herein either spelled out or abbreviated using a 1-letter code or a three letter code.

In the context of the invention, it is referred to variants of sequences (in particular CDRs). These variants typically comprise one or more amino acid substitutions. It is evident that the variant CDRs are functional variants, i.e. having amino acid sequences that may differ from the reference amino acid sequence but which differing sequence exhibits or maintains the same functional activity as the reference sequence in the context of the described heavy and/or light chain variable domain. Specifically, as used herein, the term same functional activity means that the antibody or antibody binding fragment of the invention comprising one or more variant CDRs will maintain its specificity for Aβ42, discriminate Aβ42 from other Aβ peptides, will not show Rf interference as observed with prior art Aβ42 specific antibodies (as described herein elsewhere) and/or will show a superior clinical performance for detection of amyloid positivity in an immunoassay (in particular when used in an Aβ42/Aβ40 ratio).

As used in the context of the invention, a “conservative amino acid substitution” means the substitution of an amino acid with another amino acid selected from its same physicochemical group, wherein the physicochemical groups of amino acids are
a) the nonpolar, hydrophobic amino acids consisting of Gly, Ala, Val, Leu, Ile, Phe, Tyr, Trp, and Met;
b) the polar, neutral amino acids consisting of Ser, Thr, Asn, and Gln;
c) the positively charged, basic amino acids consisting of Arg, Lys, and His, and
d) the negatively charged, acidic amino acids consisting of Asp and Glu
wherein if Cys is to be conservatively substituted, it is substituted with Ser or Ala, and
wherein if Pro is to be conservatively substituted it is substituted with Ala.

As used in the context of the invention, a “highly conservative amino acid substitution” means the following amino acid substitutions:
a) substitution of Ala with Val, Leu, Ile or Gly;
b) substitution of Arg with Lys;
c) substitution of Asn with Gln;
d) substitution of Asp with Glu;
e) substitution of Cys with Ser;
f) substitution of Gln with Asn;
g) substitution of Glu with Asp;
h) substitution of Gly with Ala;
i) substitution of His with Arg;
j) substitution of Ile with Leu, Val or Ala;
k) substitution of Leu with Ile, Val or Ala;
l) substitution of Lys with Arg;
m) substitution of Met with Leu, Ile or Val;
n) substitution of Phe with Tyr or Trp;
o) substitution of Pro with Ala;
p) substitution of Ser with Thr;
q) substitution of Thr with Ser;
r) substitution of Trp with Phe or Tyr;
s) substitution of Tyr with Phe or Trp; and
t) substitution of Val with Leu, Ile or Ala.

As used herein, the term “% sequence identity” in connection with amino acid sequences of polypeptides/peptides and/or nucleic acid sequences or nucleic acid molecules describes the number of matches of identical amino acid or nucleic acid residues of two or more aligned sequences as compared to the number of residues making up the overall length of the compared sequences (or the overall compared portions thereof). Using an alignment of two or more sequences or subsequences, the percentage of residues that are the same may be determined when the (sub)sequences are compared and aligned for maximum correspondence over a window of comparison, or over a designated region as measured using a sequence comparison algorithm as known in the art, or when manually aligned and visually inspected. Non-limiting examples of algorithms for use in determining sequence identity include, for example, those based on the NCBI BLAST algorithm (Altschul et al., Nucleic Acids Res 25(1997), 3389-3402), CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e. gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul et al., Nucl Acids Res., 25(1977), 3389).

As used herein, “nucleic acid molecule”, “nucleic acid sequence”, “polynucleotide” and analogous terms include both genomic DNA and cDNA, as well as RNA capable of driving expression of an antibody or antigen binding fragment of the invention. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. In embodiments, the nucleic acids/nucleic acid sequences of the invention may be isolated. Thus, the nucleic acid molecules may, for example, be nucleic acid molecules that have been synthesized according to conventional protocols of organic chemistry, according to recombinant methods, or produced semi-synthetically, e.g. by combining chemical synthesis and recombinant methods. The person skilled in the art is familiar with the preparation and the use of such nucleic acid molecules.

“Immunoassays” as used herein are well-established bioanalytical methods in which detection or quantitation of an analyte depends on the reaction of the analyte and at least one analyte-specific binding agent, thus forming an analyte:binding agent complex. In the context of the present invention, at least one of the at least one analyte specific binding agent is an antibody of the invention. The specific embodiment of a “sandwich” immunoassay can be used for analytes possessing more than one recognition epitopes. Thus, a sandwich assay requires at least two binding agents that attach to non-overlapping epitopes on the analyte. In a “heterogeneous sandwich immunoassay” one of the binding agents has the functional role of an analyte-specific capture binding agent; this binding agent is or (during the course of the assay) becomes immobilized on a solid phase. A second analyte-specific binding agent is supplied in dissolved form in the liquid phase. A sandwich-like complex is formed once the respective analyte is bound by a first and a second binding agent (binding agent-1:analyte:binding agent-2). The sandwich-like complex is also referred to as “detection complex”. Within the detection complex the analyte is sandwiched between the binding agents, i.e. in such a complex the analyte represents a connecting element between the first binding agent and a second binding agent.

The term “heterogeneous” (as opposed to “homogeneous”) denotes two essential and separate steps in the assay procedure. In the first step, a detection complex containing label is formed and immobilized, however with unbound label still surrounding the complexes. Prior to determination of a label-dependent signal unbound label is removed from the immobilized detection complex, thus representing the second step. In contrast, a homogeneous assay produces an analyte-dependent detectable signal by way of single-step incubation and does not require a washing step.

In a heterogeneous immunoassay the solid phase is functionalized such that it may have bound to its surface the functional capture binding agent (the first binding agent), prior to being contacted with the analyte; or the surface of the solid phase is functionalized in order to be capable of anchoring a first binding agent, after it has reacted with the analyte. In the latter case, the anchoring process must not interfere with the binding agent's ability to specifically capture and bind the analyte. A second binding agent present in the liquid phase is used for detection of bound analyte. Thus, in a heterogeneous immunoassay the analyte is allowed to bind to the first (capture) and second (detector) binding agents. Thereby a “detection complex” is formed wherein the analyte is sandwiched between the capture binding agent and the detector binding agent. In a typical embodiment, the detector binding agent is labeled prior to being contacted with the analyte; alternatively a label is specifically attached to the detector binding agent after analyte binding. With the detection complexes being immobilized on the solid phase, the amount of label detectable on the solid phase corresponds to the amount of sandwiched analyte. After removal of unbound label, immobilized label indicating presence and amount of analyte can be detected.

A “competitive immunoassay” as used herein preferably employs a single binding agent directly interacting with the analyte (i.e. Aβ42). A “competitive heterogeneous immunoassay typically detects a signal of a detection label that inversely corresponds to the amount of analyte in a sample. In preferred embodiments herein, the competitive immunoassay may be a heterogeneous competitive immunoassay. In embodiments, the sample with the analyte is mixed with an artificially produced labeled analogon of the analyte that is capable of reacting with the analyte-specific binding agent (e.g. antibody or antigen-binding fragment of the invention). In the assay, the analyte and the analogon compete for binding to a capture binding agent (e.g. the antibody of the invention) which is or becomes immobilized. The amount of binding agent is selected to be limiting in this setting. Following the binding step, the higher the amount of immobilized label, the smaller the amount of the non-labeled analyte that was capable of competing for the capture binding agent. Immobilized label is determined after a washing step. In this setting, the amount of label that is detectable on the solid phase inversely corresponds to the amount of analyte that was initially present in the sample.

The term “specific binding agent” refers to a natural or non-natural molecule that specifically binds to a target. Examples of specific binding agents include, but are not limited to, proteins, peptides and nucleic acids. In certain embodiments, a specific binding agent is an antibody or a nucleic acid. In certain embodiments, a specific binding agent is an antibody. In certain embodiments, a specific binding agent comprises the antigen binding region of an antibody. The terms target and antigen can be used interchangeably. In one embodiment the target is an antigen and the specific binding agent is an antibody. A specific binding agent has at least an affinity of 10⁷ l/mol for its corresponding target molecule. The specific binding agent in one embodiment has at least an affinity of 10⁸ l/mol or better, or in a further embodiment of at least 10¹ l/mol or better for its target molecule. In one embodiment the specific binding agent as disclosed herein is selected from the group consisting of an antibody or an aptamer. In one embodiment the specific binding agent is a specifically binding nucleic acid also known as aptamer.

The term “aptamer” refers to a nucleic acid that recognizes and binds to polypeptides. Aptamers can be isolated by selection methods such as SELEX (see e.g. Jayasena (1999) Clin. Chem., 45, 1628-50; Klug and Famulok (1994) M. Mol. Biol. Rep., 20, 97-107; U.S. Pat. No. 5,582,981) from a large pool of different single-stranded RNA molecules. Aptamers can also be synthesized and selected in their mirror-image form, for example as the L-ribonucleotide (Nolte et al. (1996) Nat. Biotechnol., 14, 1116-9; Klussmann et al. (1996) Nat. Biotechnol., 14, 1112-5). Forms which have been isolated in the later way enjoy the advantage that they are not degraded by naturally occurring ribonucleases and, therefore, possess greater stability.

In the context of the present disclosure reference is made to two prior art Aβ42 specific antibodies: 21F12 or H31L21. Both antibodies are commercially available: 21F12 from Absolute Antibody (cat. no. Ab02391-3.0) or H31L21 from ThermoFisher Scientific (cat. no. 700254). The amino acid sequence of the VH and VL of 21F12 is disclosed in WO2014/007982, which is incorporated herein in its entirety by reference. The VH domain of 21F12 has the amino acid sequence as depicted in SEQ ID NO: 19. The VL domain of 21F12 has the amino acid sequence as depicted in SEQ ID NO: 20. When referred herein to “21F12”, the term encompasses the full-length 21F12 antibody (as commercially available), any antibody or antigen-binding fragment comprising the VH and VL of 21F12 (e.g., comprising SEQ ID NOs: 19 and 20, respectively) and any Aβ42 specific antibody comprising the CDRs as comprised in SEQ ID NOs: 19 and 20.

Also referred to herein is to antibody 3D6. This antibody is not Aβ42 specific but recognizes also other Aβ peptides. It binds to the amino acids 1-5 of Aβ42. The heavy chain and light chain sequences of this antibody (as mouse antibody and as humanized antibody) have also been disclosed in EP1613347, which is incorporated herein in its entirety by reference. The 3D6 antibody is also commercially available (Creative Biolabs, cat. No. PABL-011). The VH domain of 3D6 preferably has the amino acid sequence as depicted in SEQ ID NO: 21. The VL domain of 3D6 preferably has the amino acid sequence as depicted in SEQ ID NO: 22. When referred herein to 3D6, the term encompasses the full-length 3D6 antibody (e.g. as mouse antibody or any humanized variant thereof), any antibody or antigen-binding fragment comprising the VH and VL of 3D6 (e.g., comprising SEQ ID Nos: 21 and 22, respectively) and any Aβ antibody comprising the CDRs as comprised in SEQ ID NOs: 21 and 22.

“Detectable label” as used herein relates to a label that allows for detection. According to an embodiment of the current invention a detectable label is an enzyme, or a label emitting light, in an embodiment fluorescence, luminescence, chemiluminescence, electro¬chemiluminescence or radioactivity. In a preferred embodiment the label is an electrochemiluminescent label, in an embodiment Tris(2,2′-bipyridyl)ruthenium(II)-complex (Ru(bpy)). As the interference is caused by the three-dimensional structure of the label molecule that attracts auto-antibodies and similar interfering molecules and not by the signal-emitting mechanism of said label, such as e.g. light or radioactivity, all the above-referenced labels can be used in the current invention.

Numerous detection labels (also referred to as dyes) are available which can be generally grouped into the following categories, all of them together and each of them representing embodiments according the present disclosure:

### (a) Fluorescent Dyes

Fluorescent dyes are e.g. described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oregon, USA) and Pierce Biotechnology, Inc. (Rockford, Ill.).

### (b) Luminescent Dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)3]2+(which releases a photon at ˜620 nm) regenerating with TPA (Tripropylamine) in liquid phase or liquid-solid interface are used as ECL-labels.

Recently also Iridium-based ECL-labels have been described (WO2012107419(A1)).
(c) Radioactive labels make use of radioisotopes (radionuclides), such as 3H, 11C, 14C, 18F, 32P, 35S, 64Cu, 68Gn, 86Y, 89Zr, 99TC, 111In, 123I, 124I, 125I, 131I, 133Xe, 177Lu, 211At, or 131Bi.
(d) Metal-chelate complexes suitable as labels for imaging and therapeutic purposes are well-known in the art (US 2010/0111856; U.S. Pat. Nos. 5,342,606; 5,428,155; 5,316,757; 5,480,990; 5,462,725; 5,428,139; 5,385,893; 5,739,294; 5,750,660; 5,834,456; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

A “sample” as used in the context of the present disclosure may be a liquid sample comprising or suspected to comprise Aβ42. The sample may in particular be a body fluid, such as, but not restricted to a blood sample, cerebrospinal fluid, seminal fluid, saliva or urine. In embodiments, the sample is a blood sample, such as whole blood, serum or plasma. In embodiments, the sample is serum or plasma.

The “level” of a biomarker (e.g. Aβ42 or Aβ40) in a sample means that the amount or concentration of the respective biomarker molecule in the sample is determined. The amount of a substance may be an absolute amount (e.g. give in mass) or a standards-defined quantity that measures the size of an ensemble of elementary entities, such as atoms, molecules, electrons, and other particles. It is sometimes referred to as chemical amount. The International System of Units (SI) defines the amount of substance to be proportional to the number of elementary entities present. The SI unit for amount of substance is the mole. It has the unit symbol mol. The concentration of a substance is the amount of a constituent divided by the total volume of a mixture. Several types of mathematical description can be distinguished: mass concentration, molar concentration, number concentration, and volume concentration. The term concentration can be applied to any kind of chemical mixture, but most frequently it refers to solutes and solvents in solutions. The molar (amount) concentration has variants such as normal concentration and osmotic concentration.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The word “comprise”, and variations such as “comprises” and “comprising”, is to be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

All amino acid sequences provided herein are presented starting with the most N-terminal residue and ending with the most C-terminal residue, as customarily done in the art, and the one-letter or three-letter code abbreviations as used to identify amino acids throughout the present invention correspond to those commonly used for amino acids.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

### Description of Sequences

SEQ ID NO: 1: VH sequence of antibody 3.2.52
EVQLQQSGPELVKLGASVRVSCRTSGFTITEYTMHWVKQSHGKSLEWIGG
INPITGGSRNNLKFKDKATLTGDKSSSTVYMDLRSLTSEGSTVNYCVRGR
TMGPLDYWGQGTTLTVSS
SEQ ID NO: 2: VL sequence of antibody 3.2.52
DVVMTQTPLTLSVTIGQPVSISCKSSQSLLYTNGKTFLNWLFQRPGQSPK
RLIYMVSKLESGVPDRFSGSGSGTDFTLKISRVEAEDLGIYYCMQSTHFP
LTFGGGTKLELK
SEQ ID NO: 3: CDR-H1 sequence of antibody
3.2.52 according to Kabat
EYTMH
SEQ ID NO: 4: CDR-H2 sequence of antibody
3.2.52 according to Kabat
GINPITGGSRNNLKFKD
SEQ ID NO: 5: CDR-H3 sequence of antibody
3.2.52 according to Kabat
GRTLGPLDY
SEQ ID NO: 6: CDR-L1 sequence of antibody
3.2.52 according to Kabat
KSSQSLLYTNGKTFLN
SEQ ID NO: 7: CDR-L2 sequence of antibody
3.2.52 according to Kabat
LVSKLES
SEQ ID NO: 8: CDR-L3 sequence of antibody
3.2.52 according to Kabat
LQSTHFPLT
SEQ ID NO: 9: Heavy chain sequence of
F(ab′)₂ fragment of antibody 3.2.52
EVQLQQSGPELVKLGASVRVSCRTSGFTITEYTMHWVKQSHGKSLEWIGG
INPITGGSRNNLKFKDKATLTGDKSSSTVYMDLRSLTSEGSTVNYCVRGR
TMGPLDYWGQGTTLTVSSAKTTPPSVYPLAPGCGDTTGSSVTLGCLVKGY
FPESVTVTWNSGSLSSSVHTFPALLQSGLYTMSSSVTVPSSTWPSQTVTC
SVAHPASSTTVDKKLEPSGPISTINPCPPCKECHKCP
SEQ ID NO: 10: Light chain sequence of
F(ab′)₂ fragment and full length IgG of
antibody 3.2.52
DVVMTQTPLTLSVTIGQPVSISCKSSQSLLYTNGKTFLNWLFQRPGQSPK
RLIYMVSKLESGVPDRFSGSGSGTDFTLKISRVEAEDLGIYYCMQSTHFP
LTFGGGTKLELKRADAAPTVSIFPPSSEQLTSGGASVVCFLNNFYPKDIN
VKWKIDGSERQNGVLNSWTDQDSKDSTYSMSSTLTLTKDEYERHNSYTCE
ATHKTSTSPIVKSFNRNEC
SEQ ID NO: 11 FW(HC)1 of VH of antibody 3.2.52
EVQLQQSGPELVKLGASVRVSCRTSGFTIT
SEQ ID NO: 12 FW(HC)2 of VH of antibody 3.2.52
WVKQSHGKSLEWIG
SEQ ID NO: 13 FW(HC)3 of VH of antibody 3.2.52
KATLTGDKSSSTVYMDLRSLTSEGSTVNYCVR
SEQ ID NO: 14 FW(HC)4 of VH of antibody 3.2.52
WGQGTTLTVSS
SEQ ID NO: 15 FW(LC)1 of VL of antibody 3.2.52
DVVMTQTPLTLSVTIGQPVSISC
SEQ ID NO: 16 FW(LC)2 of VL of antibody 3.2.52
WLFQRPGQSPKRLIY
SEQ ID NO: 17 FW(LC)3 of VL of antibody 3.2.52
GVPDRFSGSGSGTDFTLKISRVEAEDLGIYYC
SEQ ID NO: 18 FW(LC)4 of VL of antibody 3.2.52
FGGGTKLELK
SEQ ID NO: 19 VH sequence of antibody 21F12
EVQLQQSGPELLKPGASVKISCKASGFTFTDYTMHWMKQSHGKSLEWIGG
INPNSGGTIYNEKFKDKATLTVDKSSRTAYMELRSLTSEDSAVYFCTRGV
YDGYFYWGQGTLVTVSA
SEQ ID NO: 20 VL sequence of antibody 21F12
DVVMTQTPLSLPVSLGDQASISCRSSQSLVYSNGNTFLHWYLQKPGQSPK
LLIYKVSTRFSGVPDRFSGSGSGSDFTLKISRVEAEDLGIYFCSQTTHAP
FTFGSGTKLAIR
SEQ ID NO: 21 VH sequence of mouse 3D6
EVKLVESGGGLVKPGASLKLSCAASGFTFSNYGMSWVRQNSDKRLEWVAS
IRSGGGRTYYSDNVKGRFTISRENAKNTLYLQMSSLKSEDTALYYCVRYD
HYSGSSDYWGQGTTVTVSS
SEQ ID NO: 22 VL sequence of mouse 3D6
YVVMTQTPLTLSVTIGQPASISCKSSQSLLDSDGKTYLNWLLQRPGQSPK
RLIYLVSKLDSGVPDRFTGSGSGTDFTLKISRIEAEDLGLYYCWQGTHFP
RTFGGGTKLEIK
SEQ ID NO: 23 Heavy chain sequence of 3.2.52
EVQLQQSGPELVKLGASVRVSCRTSGFTITEYTMHWVKQSHGKSLEWIGG
INPITGGSRNNLKFKDKATLTGDKSSSTVYMDLRSLTSEGSTVNYCVRGR
TMGPLDYWGQGTTLTVSSAKTTPPSVYPLAPGCGDTTGSSVTLGCLVKGY
FPESVTVTWNSGSLSSSVHTFPALLQSGLYTMSSSVTVPSSTWPSQTVTC
SVAHPASSTTVDKKLEPSGPISTINPCPPCKECHKCPAPNLEGGPSVFIF
PPNIKDVLMISLTPKVTCVVVDVSEDDPDVRISWFVNNVEVHTAQTQTHR
EDYNSTIRVVSALPIQHQDWMSGKEFKCKVNNKDLPSPIERTISKIKGLV
RAPQVYILPPPAEQLSRKDVSLTCLVVGFNPGDISVEWTSNGHTEENYKD
TAPVLDSDGSYFIYSKLDIKTSKWEKTDSFSCNVRHEGLKNYYLKKTISR
SPGK
SEQ ID NO: 24 Aß42 amino acids 35-42
MVGGVVIA

The following examples and figures are provided to aid the understanding of the present invention, the true scope of which is set forth in the appended claims and herein above. It is understood that modifications can be made in the procedures set forth without departing from the spirit of the invention.

### DESCRIPTION OF THE FIGURES

FIG. 1 shows Kinetic profiles for mouse clone M-3.2.52 binding Aβ42. Antibody interactions with Aβ42 at 1.2 nM, 3.7 nM, 11 nM, 33 nM and 100 nM (black), overlaid with a Langmuir 1:1 binding model (grey). Despite a complex antibody Aβ-42 binding behavior at higher concentrations, the kinetic quantification was facilitated with sufficiently high precision by using a binary Langmuir model with RMAX global. The complex binding behavior can presumably attributed to a drifting baseline after the capture step of M-3.2.52.

FIG. 2 shows the results from receiver operating characteristic area under the curve (ROC AUC) analysis to investigate concordance between plasma biomarkers ratios and the amyloid status using different Aβ immunoassays. Amyloid status was determined from Elecsys® CSF pTaul81/Aβ42 ratios using a cut-off of 0.024 (113 amyloid positive, 118 amyloid negative). 95% CIs were calculated using DeLong approach. The performance of the plasma Aβ42/Aβ40 ratio using the novel 3.2.52 antibody is shown in gray (Ab42(new)/Ab40) while the Aβ42/Aβ40 ratio using the 21F12 antibody is shown in black (Ab42(old)/Ab40). DeLong test was used to calculate the p-value for comparison of both ratios.

FIG. 3 shows the results from measurement of rheumatoid factor (Rf) interference when using different Aβ42 antibodies. Plasma samples were measured in the absence of external Rf (0 IU/mL, black bars) and spiked with Rf concentrate at an amount that corresponds to 300 IU/ml Rf activity (gray bars). The percentage of concentration recovery was calculated by calculating the ratio between the determined concentration of Aβ42 in absence of external Rf and the determined concentration of Aβ42 in the presence of 300 IU/ml Rf. The obtained value is multiplied by 100 so as to achieve a percentage.

FIG. 4 shows the results from measurement of rheumatoid factor (Rf) interference over a wide concentration range when using 21F12 (black solid line) and 3.2.52 F(ab′)2 fragments (gray dashed line). The percentage of concentration recovery was calculated by calculating the ratio between the determined concentration of Aβ42 in absence of external Rf and the determined concentration of Aβ42 in the presence of Rf. The obtained value is multiplied by 100 so as to achieve a percentage.

FIG. 5 shows the effect of addition of full-length murine IgG (binding to an epitope that is not related to Aβ42) to the sample mixture on rheumatoid factor (Rf) interference when using 21F12. Three different concentrations of murine IgG were tested: 0 μg/mL (black bars), 250 μg/mL (gray bars), and 500 μg/mL (white hollow bars). Plasma samples were measured in the absence of external Rf (0 IU/mL) and spiked with Rf concentrate at an amount that corresponds to 300, 600, and 1200 IU/ml Rf activity. The percentage of concentration recovery was calculated by calculating the ratio between the determined concentration of Aβ42 in absence of external Rf and the determined concentration of Aβ42 in the presence of Rf. The obtained value is multiplied by 100 so as to achieve a percentage.

FIG. 6 shows the rheumatoid factor (Rf) interference when using 21F12 either as mouse Fab (black bars) or as chimeric mouse/human Fab (human IgG1 CH1 and CL and mouse VH and VL, white hollow bars). Plasma samples were measured in the absence of external Rf (0 IU/mL) and spiked with Rf concentrate at an amount that corresponds to 300, 600, and 1200 IU/ml Rf activity. The percentage of concentration recovery was calculated by calculating the ratio between the determined concentration of Aβ42 in absence of external Rf and the determined concentration of Aβ42 in the presence of Rf. The obtained value is multiplied by 100 so as to achieve a percentage.

FIG. 7 shows an alignment of the VHs of antibodies 21F12 and 3.2.52. CDRs are highlighted in gray.

FIG. 8 shows an alignment of the VLs of antibodies 21F12 and 3.2.52. CDRs are highlighted in gray.

FIG. 9 shows a schematic representation of the assay setup to study whether Aβ42 antibodies are bound by Rf IgM contained in native samples from patients with elevated Rf levels. Biotinylated Aβ42 antibodies were coupled to streptavidin beads, Rf IgM-containing native samples were added, and bound IgMs were detected with a ruthenylated anti-IgM antibody.

### EXAMPLES

### Example 1: Generation and Screening of a Monoclonal Anti-Aβ42 Specific Antibody

### Immunogens and Screening Reagents

For the development of the monoclonal anti-Aβ42 antibody the peptide (Aβ(Cystein-35-42)) was synthesized, comprising the amino acids 35-42 of Aβ42 peptide (SEQ: MVGGVVIA; SEQ ID NO:24), and was coupled to the carrier hemocyanin to enhance immunogenicity. Biotinylated screening reagents were synthesized corresponding to the C-terminal of the peptides Aβ40 (amino acids 33-40 of the Aβ40 peptide), Aβ42 (amino acids 1-12 and 35-42 (connected with a linker) of the Aβ42 peptide) and Aβ43 (amino acids 35-43 of the Aβ43 peptide), all comprising free C-terminus specific for the respective Aβ peptide. The C-terminus of the peptides was free and e.g. available for binding to an antibody.

### Immunization

Here we describe the development of an antibody specific for the Abeta42 peptide. For the generation of this antibody NMRI mice were immunized with the hemocyanin-(Aβ(Cystein-35-42)) peptide (Sequence: 35 MVGGVVIA 42; SEQ ID NO: 24). To enhance the immunogenicity the peptide was coupled to hemocyanin, as a carrier protein. The coupling was made such that the C-terminus of Aβ42 was free, as it was intended to obtain an antibody specifically binding to Aβ42. The mice were subjected to three immunizations. For the first immunization, 100 μg of the immunogen were emulsified in complete Freund's Adjuvant (CFA) and were administered intraperitoneally to the mice. 100 μg of the immunogen were mixed with Incomplete Freund's Adjuvant (IFA) for the two following immunizations and were administered subcutaneously or intraperitoneally, 6 weeks or 10 weeks after the start of the immunization.

### Hybridoma Generation and Antibody Screening

The immunized animals were sacrificed and the spleen cells were immortalized according to methods that are known to the expert, e.g. the hybridoma technology (Kohler and Milstein, Nature 256 (1975), 495-497). In more detail, the spleen cells of the immunized mice were mixed with P3×63Ag8-653 myeloma cells (ATTCC-CRL 8375) in a ratio of 1:5 and centrifuged. The cells were again washed, the cell sediment was loosened and 1 ml PEG (MG 4000, Merck) was added and passed through a pipette. After 1 minute in a water-bath, 25 ml of RPMI 1640 base medium were added drop-wise at room temperature and over a period of 10 minutes; the solution was mixed and incubated for 30 min a 37° C. and 5% CO₂. After centrifugation, the sedimented cells were placed into 24-well cell culture plates with 5×10″ spleen cells per well in 1 ml selection medium (100 mM hypoxanthine, 1 g/ml azaserine in RPMI 1640+10% FCS). After 10 days, these primary cultures were tested for specific antibody synthesis.

For the so-called Hit-ELISA the respective biotinylated screening peptides (amino acids 1-12 and 35-42 (connected with a linker) of the Aβ42 peptide) were immobilized on the surface of streptavidin coated 96-well plates at a concentration of 200 ng/ml. The peptide solution was incubated on the plates for 60 min at RT. After washing the plates were blocked with 0.5% (w/v) Byco C for 30 min at room temperature to reduce background signals. The plates were washed again and 30 μl of the hybridoma culture were transferred to the 96 well plates and incubated for 1 h at RT. For the detection of the antibodies bound to the screening peptides, Peroxidase-conjugated AffiniPure F(ab′)2 Fragment Anti-Mouse IgG and ABST (Roche) as a substrate were added.

The Hit ELISA revealed 1508 antibodies that recognized the Aβ42 mimicking peptide (amino acids 1-12 and 35-42 (connected with a linker) of the Aβ42 peptide).

Primary cultures of the corresponding specificity were evaluated by Biacore and the clones with good kinetic characteristics, including the clone 3.2.52 (as shown in Example 2 below), were cloned via FACS (cell sorter) in 96-well cell culture plates. Interleukin-6 (Boehringer Mannheim, Cat. No. 1271172, 100 U/ml) was added to the medium as a growth additive. All the clones were re-evaluated by ELISA for specificity, the concentration and IgG subclass were determined.

Due to its kinetic features and Aβ42 specificity (see Example 2, below) one of the selected antibodies was clone 3.2.52.

For further experiments, IgG was purified from culture supernatant via one-step protein A affinity purification (HiTrap MabSelect SuRe, GE) according to supplier's instructions.

### Example 2: Characterization of the Aβ42 Antibody 3.2.52

A described in Example 1 above, one of the clones selected as specific Aβ42 binder was clone 3.2.52. This clone was further characterized to evaluate its suitability in a sandwich immunoassay for specifically detecting Aβ42, especially in blood samples.

### Kinetic Characterization

Mouse anti Aβ-42 antibody 3.2.52 (IgG2b), expressed in hybridoma, was further characterized.

Measurements were performed using the BIAcore™ 8K+ instruments. Aβ-42 concentration series ranging from 1.2 nM to 900 nM were injected at 30 L/min. The association phase was monitored for 3 min, the dissociation phase between 5 min to 10 min at 37° C.

The measurements were performed using PBS-DT+, pH7.4 (10 mM phosphate buffer, 2.7 mM KCl, 137 mM NaCl. 5% DMSO, 0.05% Tween 20) as running buffer.

The kinetic rate constants and the dissociation equilibrium constants K_{D} were calculated using a Langmuir 1:1 fit model according Scrubber Version 2.0.c.

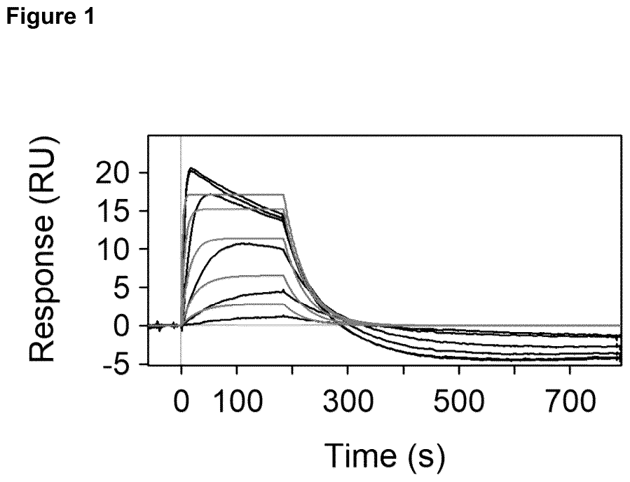

The measurements are shown in FIG. 1.

37° C. kinetics of the in hybridoma expressed mouse clone M-3.2.52: kₐ=3.8±0.1*10⁶ M⁻¹s⁻¹, k_{d}=2.53±0.06*10⁻² s⁻¹, t_{/2 diss}<1 min, MR=0.7, Affinity K_{D}=6.7±0.2 nM. These kinetic characteristics of antibody 3.2.52 proposes that the antibody is well suitable for use in an Aβ42 sandwich immunoassay, especially using the Elecsys® system.

### Assessment of Cross-Reactivity Via Elecsys

Abeta38 (amino acids 1-12 and 25-38 (connected with linker) of the Aβ38 peptide), Abeta40 (amino acids 1-12 and 25-40 (connected with linker) of the Aβ40 peptide) and

| | Abeta38 | | Abeta40 | | Abeta43 | |
|---|---|---|---|---|---|---|
| Analyte excess | Abeta42 | Recovery | Abeta42 | Recovery | Abeta42 | Recovery |
| over normal | Plasma | to 0x | Plasma | to 0x | Plasma | to 0x |
| level in healthy | Conc. | excess | Conc. | excess | Conc. | excess |
| patients | [pg/mL] | [%] | [pg/mL] | [%] | [pg/mL] | [%] |
| 0x | 33.8 | 100 | 33.7 | 100 | 33.5 | 100 |
| 2x | 33.3 | 99 | 33.1 | 98 | 33.9 | 101 |
| 10x | 33.8 | 100 | 33.8 | 100 | 33.0 | 99 |
| 50x | 33.3 | 99 | 33.9 | 101 | 32.8 | 98 |

Abeta43 (amino acids 1-12 and 34-43 (connected with linker) of the Aβ43 peptide) peptides were spiked in 2×, 10×, and 50× excess over the natural expected concentration (Abeta38 expected level in healthy patients: 25 μg/mL; Abeta40: 250 μg/mL; Abeta43: 4 μg/mL) into a single plasma sample. Truncated Abeta peptides were used in this experiment since full-length Abeta peptides aggregate and stick to tube walls, which makes the results more unreliable. The impact on the determined Abeta42 level as determined by Elecsys® using 3.2.52 as the capture antibody and using a Aβ (not Aβ42 specific) antibody binding to the N-terminus (amino acids 1-5 of Aβ42) but not competing for binding with 3.2.52 as detection antibody (3D6, EP1613347) was assessed. The results are depicted in Table 1. The results confirm that the antibody 3.2.52 is specifically binding to Aβ42.

Table 1 shows the results from the assessment of 3.2.52 cross-reactivity with Abeta38, Abeta40, and Abeta43 as determined via Elecsys (using 3.2.52 as the capture antibody). Concentration recovery was normalized against the unspiked samples. All three peptides can be spiked up to 50× excess over the natural expected concentration with no effect on concentration recovery, indicating that no relevant cross-reactivity to Aβ38, Aβ40, or Aβ43 can be observed in native plasma samples.

### Example 3: Novel Aβ42 Antibody 3.2.52 Improves Detection of Amyloid Status in Plasma Samples Compared to Prior Art Antibodies

Cerebrospinal fluid and plasma samples collected from 240 patients (35 cognitively normal, 110 mild cognitive impairment, 95 Alzheimer's Disease [MMSE >22]) were analyzed with the following Elecsys® plasma assays: Aβ42 using 21F12 as the capture antibody, Aβ42 using 3.2.52 as the capture antibody, and Aβ40. 21F12 is an Aβ42 antibody specifically recognizing Aβ42 but not other Aβ peptides frequently used in prior art Aβ42 assays. The Elecsys method is an antibody-based technique based on one capture and one detection antibody. The general Elecsys assay setup was as follows: Up to 50 μL of sample, a biotinylated monoclonal antibody (capture antibody) and a monoclonal antibody labeled with a ruthenium complex (detection antibody; for details on antibodies see table 2 below) were first co-incubated for 9 minutes to form a sandwich complex comprising the biotinylated antibody, analyte and the ruthenylated antibody. In the second incubation step (9 minutes), streptavidin-coated microparticles (Elecsys beads) were added to the mixture of the first incubation step and, as a result, the complex comprising the biotinylated antibody, analyte and the ruthenylated antibody became bound to the solid phase via interaction of biotin and streptavidin. The reaction mixture was aspirated into the measuring cell where the microparticles were magnetically captured onto the surface of the electrode. Unbound substances were then removed with ProCell/ProCell M. Application of a voltage to the electrode then induced chemiluminescent emission was measured by a photomultiplier. Sample concentrations were determined from a 5-point calibration curve.

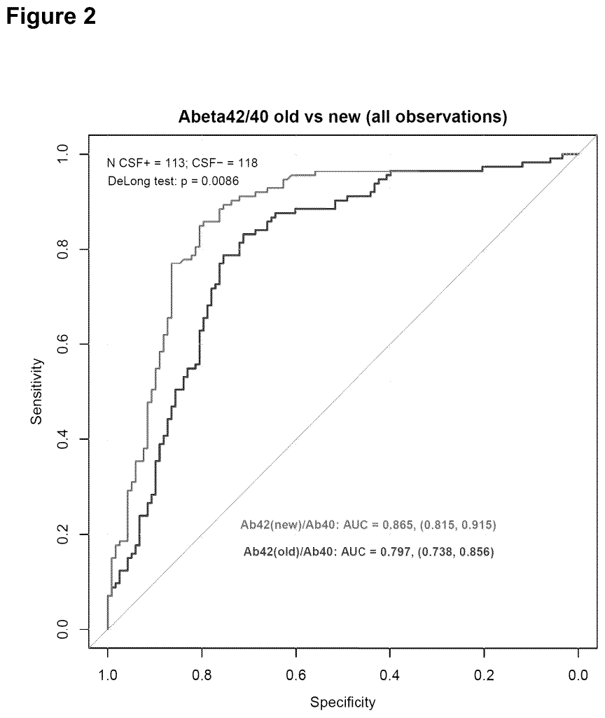

Amyloid status was determined from Elecsys® CSF pTaul81/Aβ42 ratios using a cut-off of 0.024 (data available for 231 patients; 113 amyloid positive, 118 amyloid negative). Receiver operating characteristic area under the curve (ROC AUC) analysis was used to investigate concordance between plasma biomarkers ratios and the amyloid status as determined by Elecsys® CSF pTaul81/Aβ42. 95% CIs were calculated using DeLong approach. The plasma Aβ42/Aβ40 ratio, which has been shown to be a useful diagnostic marker for amyloid positivity in multiple studies (e.g. reviewed in Hampel et al., Nat Rev Neurol, 2018. 14(11): p. 639-652), using the novel 3.2.52 antibody had surprisingly a significantly higher AUC (0.865, 95% CI0.815-0.915) compared with the Aβ42/Aβ40 ratio using the 21F12 antibody (0.797, 95% CI 0.738-0.856; p=0.0086) for discriminating Aβ-positive (also referred to herein as amyloid positive) vs -negative participants (see FIG. 2).

This result demonstrates that the novel Aβ42 specific antibody as provided herein has surprising and unexpected features that apparently improve the clinical outcome of Aβ42 assays in plasma compared to prior art Aβ42 antibodies.

### Example 4: Prior Art Monoclonal Anti-Aβ42 Antibodies Show an IgM Mediated Rheumatoid Factor Interference in Aβ42 Immunoassays that is Overcome by the New Aβ42 Antibody

One factor that is important for a reliable and robust detection of Aβ42 especially in blood samples, such as plasma samples, is that interferences between components in the sample and the immunoassay components, such as the antibodies, are kept at a minimum. A potential immunoassay interference known in the art for blood samples is the so-called rheumatoid factor (Rf) interference. Rheumatoid factors are autoantibodies, most often of the immunoglobulin M (IgM) subtype, that are commonly found in elevated concentrations in blood from patients suffering from rheumatoid arthritis and other diseases such as lupus or sepsis. Importantly, Rf are also often elevated in healthy elderly people (>70 y) (Francesca Ingegnoli et al., Dis Markers. 2013; 35(6): 727-734). Rf interference may cause false-positive or false-negative assay results and thereby lower the clinical value of such assays.

As demonstrated below, we have surprisingly found that two Aβ42-specific antibodies widely and successfully used in Aβ42 immunoassays in cerebrospinal fluid (CSF) samples (21F12 from Eli Lilly and H31L21 from ThermoFisher Scientific) suffer from a Rf interference in a number of plasma samples with increased Rf level. This previously unknown feature of these antibodies, probably due to the use of CSF samples, in which Rf interference is not an issue, seems to be a common issue of both previously known anti-Aβ42 antibodies even so they are likely structurally not related.

Strikingly, as demonstrated below, the novel Aβ42 antibody 3.2.52 as provided herein, does not show the Rf interference as observed with the other two commonly used Aβ42 antibodies widely used in the community. Due to the absence of the Rf interference the novel antibody 3.2.52 provided herein provides a more reliable and accurate quantification of Aβ42 levels in blood. As evident from Example 3 above, the antibody 3.2.52 also provided for an improved correlation of a plasma based immunoassay with standard of care CSF based immunoassays for detection of amyloid positivity. The reduced interference is an important factor among others contributing to this improved clinical performance.

### Methods

1) Spiking of Aβ42-Containing Blood Plasma Samples with Concentrated Rf

To evaluate and to compare a potential influence of Rf interference on immunoassays using different Aβ42 antibodies (i.e. 3.2.52, 21F12 and H31L21) blood plasma samples were spiked with a Rf concentrate (comprising Rf IgM) and the Aβ42 levels were detected in the presence and absence of Rf concentrate. The Rf concentrate was obtained by purification of Rf from pools of plasma from patients suffering from rheumatoid arthritis (RA), essentially following the procedures as described in J Immunol Methods, 1993,158(1):1-4, doi: 10.1016/0022-1759(93)90252-3. In brief, plasma from RA patients is first centrifuged to remove aggregated proteins. Next, the pH of supernatant is adjusted to 4.75 using 2 M acetic acid and after incubation for 60 min at RT re-adjusted to 7.5 using 2 M Tris base. After centrifugation, the supernatant is recalcified using 2.5 M CaCl₂×2H2O (final conc 20 mM). After 2 h incubation at RT, the mixture is centrifuged and the supernatant is delipified by addition of 100 mL 3.53% dextrane sulfide, 4.47 M CaCl₂) solution per L plasma. After 30 min incubation at RT, the mixture is centrifuged and the supernatant is kept. To enrich Rf IgMs, immunoglobulins are precipitated using polyethylene glycol (PEG) and further purified via ammonium sulfate precipitation in order to eliminate residual PEG. The final pellet is dissolved in 50 mM potassium phosphate, 150 mM NaCl buffer. Rf activity is determined using Roche RF-II assay.

The Elecsys® immunoassays were essentially identical except for using the different Aβ42 specific antibodies to be tested. The Elecsys® method is an antibody-based technique based on one capture and one detection antibody. The general Elecsys assay setup was as follows: Up to 50 μL of sample, a biotinylated monoclonal antibody (capture antibody) and a monoclonal antibody labeled with a ruthenium complex (detection antibody; for details on antibodies see Table 2 below) were first co-incubated for 9 minutes to form a sandwich complex comprising the biotinylated antibody, analyte and the ruthenylated antibody. In the second incubation step (9 minutes), streptavidin-coated microparticles (Elecsys beads) were added to the mixture of the first incubation step and, as a result, the complex comprising the biotinylated antibody, analyte and the ruthenylated antibody became bound to the solid phase via interaction of biotin and streptavidin. The reaction mixture was aspirated into the measuring cell where the microparticles were magnetically captured onto the surface of the electrode. Unbound substances were then removed with ProCell/ProCell M. Application of a voltage to the electrode then induced chemiluminescent emission was measured by a photomultiplier. Sample concentrations were determined from a 5-point calibration curve.

The antibodies used in the different immunoassays tested were as summarized in Table 2 below

TABLE 2
Elecsys ® immunoassay setups.

| | Biotinylated | Ruthenylated |
|---|---|---|
| Assay | antibody clone | antibody clone |
| Aβ42 assay using 3.2.52 | 3.2.52 | 3D6 |
| Aβ42 assay using 21F12 | 21F12 | 3D6 |
| variant | | |
| Aβ42 using H31L21 | H31L21 | 3D6 |
| Aβ40 | 23C2 | 3D6 |

In a first experiment, plasma samples were measured in the absence of external Rf and spiked with Rf concentrate at an amount that corresponds to 300 IU/ml Rf activity. The percentage of concentration recovery was calculated by calculating the ratio between the determined concentration of Aβ42 in absence of external Rf and the determined concentration of Aβ42 in the presence of 300 IU/ml Rf. The obtained value is multiplied by 100 so as to achieve a percentage. In this experiment, the antibodies were used in an IgG format.

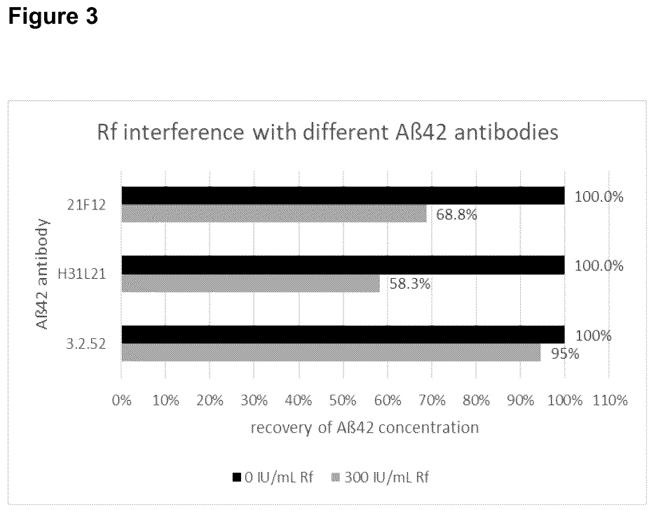

As shown in FIG. 3, very surprisingly, both widely used Aβ42 antibodies 21F12 and H31L21 deliver abnormal results in presence of 300 IU/mL Rf activity. This problem that appears to be a generic problem of the prior art antibodies was strikingly overcome by the new anti-Aβ42 antibody 3.2.52.

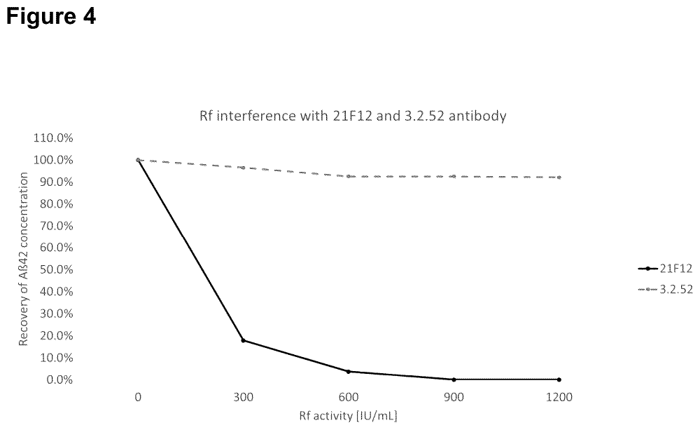

A second experiment, using another batch of Rf concentrate, compared the Rf interference on 21F12 and 3.2.52 F(ab′)2 fragments over a wide concentration range. As shown in FIG. 4, this second experiment confirmed that 3.2.52 is not affected by Rf levels up to 1200 IU/mL, while the existing antibody clone 21F12 is severely affected, causing false negative results. Notably, the interference was even more severe for the batch of Rf concentrate used in this second experiment. This suggests that the concentration of the specific Rf causing the interference is higher in the second Rf concentrate batch.

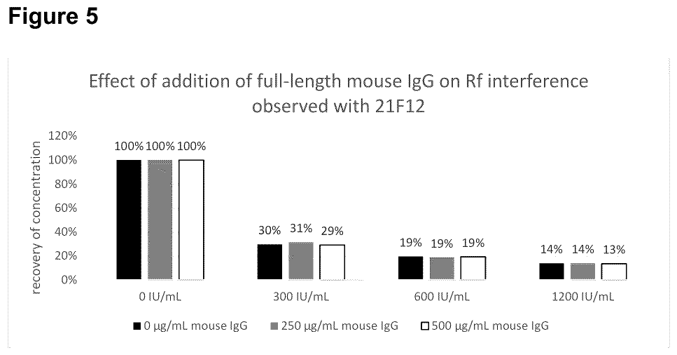

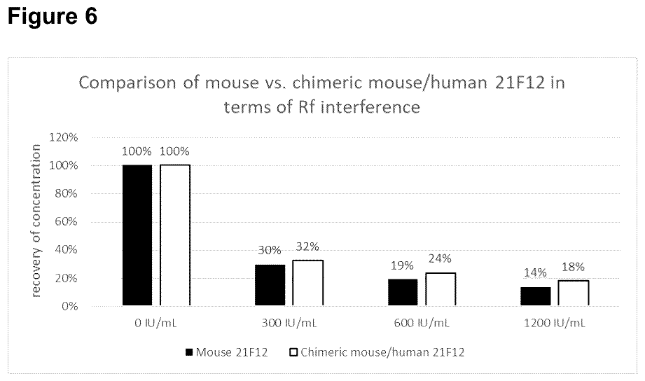

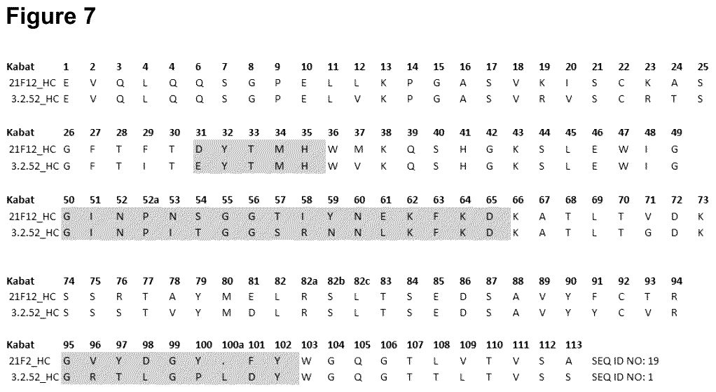

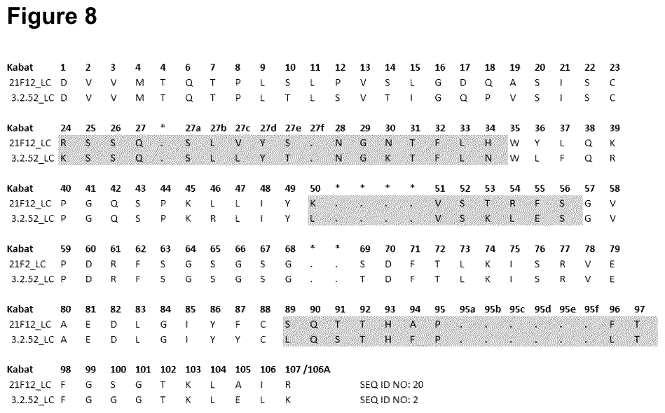

The fact that the interference was also found when using F(ab′)₂ fragments further demonstrates that the Rf interference is not a typical Rf interference targeting the Fc part of the antibody. Instead, the interference is caused by the F(ab′)₂ fragment of the 21F12 antibody. More specifically, there is strong evidence that the Rf interference is caused by the VH and VL of 21F12 (and H31L21). First, 21F12 and 3.2.52 are both murine antibodies, i.e. share a high sequence identity outside the VH and VL. Second, adding up to 500 μg/mL full length murine IgG binding to an epitope that is unrelated to Aβ42 to the sample mixture did not change the observed interference (FIG. 5). Third, producing a recombinant chimeric version of 21F12 Fab having constant regions of human IgG1 (i.e. human IgG1 CH1 and CL and mouse VH and VL) did not solve the interference (FIG. 6). Fourth, the fact that the measured Aβ concentration using 21F12 and H31L21 is lower than it should be, indicates that the Rf may interfere with antigen binding; i.e. is directed to the antigen-binding domains of 21F12 and H31L21, respectively. Especially the first and the second argument mentioned above further indicate that the CDRs of 21F12 are the major contributor to the Rf interference, as also in the framework regions of VH and VL there is an extremely high sequence identity between murine antibodies. The high sequence identity of the framework regions is evident from a comparison of the VH and VL framework sequences of 21F12 (SEQ ID NOs: 19 and 20, respectively) and 3.2.52 (SEQ ID NOs: 1 and 2, respectively) as shown in FIGS. 7 and 8. Accordingly, in sum the observed Rf interference appears to be caused by the CDRs of 21F12 (and likely also H31L21).

### 2) Quantification of Aβ42 Levels in Native Samples Containing Elevated Rf Levels.

To assess whether the striking difference in Rf interference between the previously known anti-Aβ42 antibodies and the newly generated anti-Aβ42 antibody 3.2.52 can also be observed in native serum samples, a set of serum samples with elevated Rf levels of 300 IU/mL or higher were subjected to immunoassays. In this experiment again F(ab′)2 fragments of the Aβ42 specific antibodies were used.

As shown in Table 3 below, and in line with the analysis of the spiked samples above, 3.2.52 detects higher Aβ42 levels in a subset of samples (highlighted in Table 3 in bold), suggesting that 21F12 delivered abnormally low results. This finding provides clear evidence that also in a significant subset of serum samples with elevated Rf, a significant Rf interference leading to an abnormally low Aβ42 level is found for the previously available antibodies (exemplified by 21F12). By contrast, the novel antibody 3.2.52 overcomes this shortcoming.

TABLE 3
Quantification of Aβ42 levels in native samples containing
elevated Rf levels. Levels were determined via Elecsys using
either 21F12 or 3.2.52 as capture antibodies. Concentration
recoveries (in %) were normalized against 21F12 results. Results
highlighted in gray/bold show a recovery ≥120%. or n.a.
(not available) means that the recovery could not be calculated
since the Aβ42 level determined with 21F12 was zero.

| | | | |
|---|---|---|---|
| | | Aβ42 concentration | |
| Sample | Rf | [pg/mL] | % |

| no | [IU/mL] | 21F12 | 3.2.52 | recovery |
|---|---|---|---|---|
| RF_2_31 | 457 | 12.8 | 12.0 | 93% |
| RF_2_32 | 655 | 21.2 | 17.1 | 81% |
| RF_2_33 | 377 | 31.5 | 28.9 | 92% |
| RF_3_1 | 430 | 3.29 | 3.53 | 107% |
| RF_{—}3_{—}2 | 401 | 4.46 | 29.8 | 669% |
| RF_3_3 | 394 | 13.2 | 10.7 | 81% |
| RF_{—}3_{—}4 | 321 | 16.4 | 19.8 | 121% |
| RF_3_5 | 402 | 8.55 | 6.67 | 78% |
| RF_3_6 | 371 | 22.2 | 18.6 | 84% |
| RF_3_7 | 318 | 17.2 | 18.4 | 107% |
| RF_3_8 | 402 | 20.1 | 18.8 | 93% |
| RF_3_9 | 439 | 7.29 | 6.76 | 93% |
| RF_3_10 | 322 | 28.8 | 26.0 | 90% |
| RF_{—}3_{—}11 | 383 | 19.3 | 23.4 | 122% |
| RF_3_12 | 457 | 10.6 | 8.18 | 77% |
| RF_3_13 | 1492 | 13.8 | 13.0 | 94% |
| D4 | 566 | 0.0000 | 3.96 | n.a. |
| C9 | 640 | 1.79 | 2.76 | 154% |
| D8 | 572 | 9.04 | 6.93 | 77% |
| A7 | 1664 | 0.0000 | 2.02 | n.a. |
| B6 | 386 | 2.44 | 12.1 | 496% |
| E2 | 423 | 0.203 | 2.66 | 1309% |
| E8 | 339 | 0.0000 | 4.11 | n.a. |
| A5 | 466 | 1.65 | 4.73 | 286% |
| 3950854981 | 355 | 1.90 | 4.47 | 235% |

3) Rf IgM of Native Samples with Elevated Rf Levels Binds to 21F12

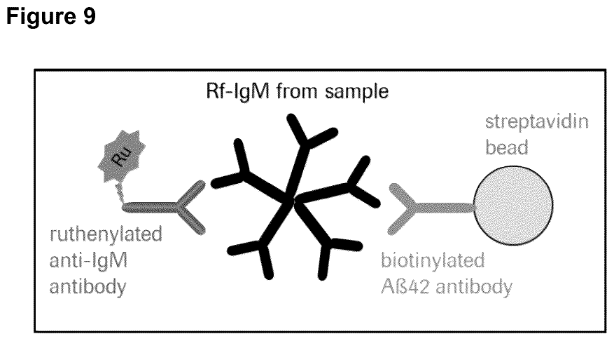

To further assess the nature of the observed Rf interference, it was assessed whether a Rf IgM is found to bind to the antibody 21F12 in the samples with elevated Rf levels (>300 IU/mL). To this end, an Elecsys assay setup was used (general setup see above), which allows studying whether Aβ42 antibodies are bound by Rf IgM contained in native samples from patients with elevated Rf levels. In brief, biotinylated Aβ42 antibodies as F(ab′)₂ fragments were coupled to streptavidin beads, Rf IgM-containing native samples are added, and bound IgMs were detected with a ruthenylated anti-IgM antibody. A schematic representation of the assay setup is depicted in FIG. 9 and is referred to herein also as μ-capture assay.

Using this assay format, we compared binding of native Rf-IgM to the Aβ42 antibodies 21F12 and 3.2.52 (both as F(ab′)₂ fragments) and to an unrelated control antibody TU2 (see Table 4). Binding was defined as a signal >40,000 Elecsys counts as the negative control antibody TU2 yielded a signal of maximum 39,301 counts (sample RF_3_13). Binding to 21F12 was detected in 7/24 samples (29.2%) with elevated Rf levels, while no binding could be detected for 3.2.52 and TU2 (see Table 4).

TABLE 4
Assessment of binding of Aβ42 antibodies 21F12 and 3.2.52 (both as
F(ab′)2 fragments) to Rf IgM contained in native samples from patients
with elevated Rf levels via Elecsys. TU2 was used as an unrelated
negative control antibody. Binding was defined as a signal >40,000
Elecsys counts (highlighted in gray/bold). n.a. (not available)
means that no results were obtained for that particular sample.

| | | Elecsys counts | | |
|---|---|---|---|---|
| Sample | Rf | | | TU2 |
| no | [IU/mL] | 21F12 | 3.2.52 | (neg. ctrl.) |
| RF_2_31 | 457 | 27282 | 3702 | 2291 |
| RF_2_32 | 655 | 4225 | 3256 | 2846 |
| RF_2_33 | 377 | 9427 | 6570 | 7936 |
| RF_3_1 | 430 | 19675 | 9109 | 19318 |
| RF_{—}3_{—}2 | 401 | 281502 | 11065 | 10025 |
| RF_3_3 | 394 | 5849 | 5435 | 7432 |
| RF_{—}3_{—}4 | 321 | 99589 | 4489 | 3689 |
| RF_3_5 | 402 | 10424 | 3259 | 3161 |
| RF_3_6 | 371 | 24457 | 7244 | 10554 |
| RF_3_7 | 318 | 7581 | 5667 | 6357 |
| RF_3_8 | 402 | 6824 | 5423 | 2925 |
| RF_3_9 | 439 | 14490 | 5396 | 4268 |
| RF_3_10 | 322 | 8616 | 6284 | 7163 |
| RF_{—}3_{—}11 | 383 | 69302 | 3494 | 19243 |
| RF_3_12 | 457 | 5674 | 4150 | 1932 |
| RF_3_13 | 1492 | 4744 | 2034 | 39301 |
| D4 | 566 | 13314 | 2519 | 1316 |
| C9 | 640 | 8241 | 5686 | 4746 |
| D8 | 572 | 242034 | 11803 | 3736 |
| A7 | 1664 | n.a. | n.a. | n.a. |
| B6 | 386 | 103200 | 3375 | 23052 |
| E2 | 423 | 102294 | 4325 | 4738 |
| E8 | 339 | 117636 | 5051 | 2156 |
| A5 | 466 | 11614 | 2565 | 1796 |
| 3950854981 | 355 | n.a. | n.a. | n.a. |

Table 5 below shows a summary of the results of the experiments given in section 2) and 3). 6 samples showed consistent results across both experiments, i.e. a higher Aβ42 concentration when using the 3.2.52 antibody and also binding to the 21F12 but not 3.2.52 antibody. This confirms that IgM Rf are likely responsible for the observed Rf interference of the prior art Aβ42 antibodies. In the other samples showing the Rf interference (i.e. in which a higher concentration using 3.2.52 was detected) likely the IgM levels are too low to be detected or antibodies of other isotype, e.g. IgG cause the interference.

TABLE 5
Summary of the results of the experiments of Tables 3 and 4 to
show that prior art monoclonal anti-Aβ42 antibodies show an
IgM mediated rheumatoid factor interference in Aβ42 immunoassays
that is overcome by the new Aβ42 antibody 3.2.52. x indicates
that that particular sample gave a positive result in the respective
experiment. n.a. indicates that no results were obtained for
that particular sample/experiment. 6 samples showed consistent
results across both experiments (highlighted in gray/bold), i.e.
a higher Aβ42 concentration when using the 3.2.52 antibody
and also binding to the 21F12 but not 3.2.52 antibody.

| Sample | Rf | Higher concentration | IgM Binding to |
|---|---|---|---|
| no | [IU/mL] | with 3.2.52 | 21F12 |
| RF_2_31 | 457 | | |
| RF_2_32 | 655 | | |
| RF_2_33 | 377 | | |
| RF_3_1 | 430 | | |
| RF_{—}3_{—}2 | 401 | x | x |
| RF_3_3 | 394 | | |
| RF_{—}3_{—}4 | 321 | x | x |
| RF_3_5 | 402 | | |
| RF_3_6 | 371 | | |
| RF_3_7 | 318 | | |
| RF_3_8 | 402 | | |
| RF_3_9 | 439 | | |
| RF_3_10 | 322 | | |
| RF_{—}3_{—}11 | 383 | x | x |
| RF_3_12 | 457 | | |
| RF_3_13 | 1492 | | |
| D4 | 566 | x | |
| C9 | 640 | x | |
| D8 | 572 | | x |
| A7 | 1664 | x | n.a. |
| B6 | 386 | x | x |
| E2 | 423 | x | x |
| E8 | 339 | x | x |
| A5 | 466 | x | |
| 3950854981 | 355 | x | n.a. |

1. A monoclonal antibody or an antigen-binding fragment thereof specifically binding to Aβ42, comprising a heavy chain variable domain (VH) comprising (a) a CDR-H1 comprising the amino acid sequence of SEQ ID NO: 3 or a variant thereof with one amino acid substitution, (b) a CDR-H2 comprising the amino acid sequence of SEQ ID NO: 4 or a variant thereof with one amino acid substitution, and (c) a CDR-H3 comprising the amino acid sequence of SEQ ID NO: 5 or a variant thereof with one amino acid substitution, and wherein the light chain variable domain (VL) comprises (d) a CDR-L1 comprising the amino acid sequence of SEQ ID NO: 6 or a variant thereof with one amino acid substitution, (e) a CDR-L2 comprising the amino acid sequence of SEQ ID NO: 7 or a variant thereof with one amino acid substitution, and (f) a CDR-L3 comprising the amino acid sequence of SEQ ID NO: 8 or a variant thereof with one amino acid substitution.

2. The monoclonal antibody or antigen-binding fragment of claim 1, wherein the heavy chain variable domain (VH) comprises (a) CDR-H1 of the amino acid sequence of SEQ ID NO: 3, (b) CDR-H2 of the amino acid sequence of SEQ ID NO: 4, and (c) CDR-H3 of the amino acid sequence of SEQ ID NO: 5, and a light chain variable domain (VL) comprising (d) CDR-L1 of the amino acid sequence of SEQ ID NO: 6, (e) CDR-L2 of the amino acid sequence of SEQ ID NO: 7, and (f) CDR-L3 of the amino acid sequence of SEQ ID NO: 8.

3. The monoclonal antibody or the antigen-binding fragment of claim 1, wherein
(i) the heavy chain variable domain (VH) comprises the amino acid sequence of SEQ ID NO: 1 or an amino acid sequence having a sequence identity of at least 70% with SEQ ID NO: 1; and
(ii) the light chain variable domain (VL) comprises the amino acid sequence of SEQ ID NO: 2 or an amino acid sequence having a sequence identity of at least 70% with SEQ ID NO: 2.

4. The monoclonal antibody or the antigen-binding fragment of claim 1, wherein the heavy chain comprises or consists of the amino acid sequence of SEQ ID NO: 9 or an amino acid sequence having a sequence identity of at least 70% with SEQ ID NO: 9; and/or wherein the light chain comprises the amino acid sequence of SEQ ID NO: 10 or an amino acid sequence having a sequence identity of at least 70% with SEQ ID NO: 10.

5. The monoclonal antibody or the antigen-binding fragment of claim 1, wherein when the monoclonal antibody or the antigen-binding fragment is used as an Aβ42 specific binding agent in a sandwich immunoassay for detecting and/or quantifying Aβ42 in a blood sample, the accuracy for determining amyloid positivity in blood samples of a reference population with known amyloid status using an Aβ42/Aβ40 ratio is higher than for the same Aβ42 sandwich immunoassay using Aβ42 antibodies 21F12 and/or H31L21, wherein the accuracy is assessed by the area under the curve (AUC) of a receiver operating characteristic (ROC) analysis.

6. The monoclonal antibody or the antigen-binding fragment of claim 1, wherein when the monoclonal antibody or the antigen-binding fragment is used as an Aβ42 specific binding agent in an immunoassay for detecting and/or quantifying Aβ42 in a blood sample, does not show rheumatoid factor (Rf) interference, wherein the blood sample shows Rf interference with the Aβ42 antibodies 21F12 and/or H31L21 when used in an immunoassay.

7. A polynucleotide or a set of polynucleotides encoding
(i) the heavy chain or heavy chain variable domain of the monoclonal antibody or antigen binding fragment according to claim 1, and/or
(ii) the light chain or light chain variable domain of the monoclonal antibody or antigen binding fragment according to claim 1.

8. A vector comprising the polynucleotide or set of polynucleotides according to claim 7.

9. A host cell comprising the polynucleotide or set of polynucleotides according to claim 7.

10. A method of producing the monoclonal antibody or the antigen-binding fragment of claim 1, said method comprising culturing a host cell and isolating said antibody.

11. A composition comprising the antibody or antigen-binding fragment according to claim 1.

12. (canceled)

13. An in vitro method for determining the presence and/or the level of Aβ42 in a sample, said method comprising:
(a) contacting the antibody or the antigen binding fragment according to claim 1 with the sample under conditions allowing the binding of the antibody or the antigen binding fragment of claim 1 to Aβ42 in the sample; and
(b) determine the presence or level of Aβ42 in the sample by detecting the binding of the antibody or the antigen binding fragment according to claim 1 to Aβ42.

14. An in vitro method for aiding in the detection of amyloid positivity in a subject, said method comprising
a) determining the level of Aβ42 in the sample using the antibody or antigen-fragment according to claim 1;
b) determining the level of Aβ40 in the sample;
c) determining a combined value from the level of Aβ42 and the level of Aβ40; and
d) comparing the combined value to a predetermined reference combined value, wherein an altered combined value is indicative for amyloid positivity.

15. A kit for detecting and/or quantifying Aβ42 in a sample, said kit comprising the antibody or antigen binding fragment of claim 1.


SEQUENCE LISTING (24 sequences)

SEQ ID NO: 1 - protein, 118 aa, synthetic construct
  REGION 1..118: note = VH sequence of antibody 3.2.52
EVQLQQSGPELVKLGASVRVSCRTSGFTITEYTMHWVKQSHGKSLEWIGGINPITGGSRNNLKFKDKATLTGDKSSSTVYMDLRSLTSEGSTVNYCVRGRTMGPLDYWGQGTTLTVSS

SEQ ID NO: 2 - protein, 112 aa, synthetic construct
  REGION 1..112: note = VL sequence of antibody 3.2.52
DVVMTQTPLTLSVTIGQPVSISCKSSQSLLYTNGKTFLNWLFQRPGQSPKRLIYMVSKLESGVPDRFSGSGSGTDFTLKISRVEAEDLGIYYCMQSTHFPLTFGGGTKLELK

SEQ ID NO: 3 - protein, 5 aa, synthetic construct
  REGION 1..5: note = CDR-H1 sequence of antibody 3.2.52 according to Kabat
EYTMH

SEQ ID NO: 4 - protein, 17 aa, synthetic construct
  REGION 1..17: note = CDR-H2 sequence of antibody 3.2.52 according to Kabat
GINPITGGSRNNLKFKD

SEQ ID NO: 5 - protein, 9 aa, synthetic construct
  REGION 1..9: note = CDR-H3 sequence of antibody 3.2.52 according to Kabat
GRTLGPLDY

SEQ ID NO: 6 - protein, 16 aa, synthetic construct
  REGION 1..16: note = CDR-L1 sequence of antibody 3.2.52 according to Kabat
KSSQSLLYTNGKTFLN

SEQ ID NO: 7 - protein, 7 aa, synthetic construct
  REGION 1..7: note = CDR-L2 sequence of antibody 3.2.52 according to Kabat
LVSKLES

SEQ ID NO: 8 - protein, 9 aa, synthetic construct
  REGION 1..9: note = CDR-L3 sequence of antibody 3.2.52 according to Kabat
LQSTHFPLT

SEQ ID NO: 9 - protein, 237 aa, synthetic construct
  REGION 1..237: note = Heavy chain sequence of F(ab')2 fragment of antibody 3.2.52
EVQLQQSGPELVKLGASVRVSCRTSGFTITEYTMHWVKQSHGKSLEWIGGINPITGGSRNNLKFKDKATLTGDKSSSTVYMDLRSLTSEGSTVNYCVRGRTMGPLDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCP

SEQ ID NO: 10 - protein, 219 aa, synthetic construct
  REGION 1..219: note = Light chain sequence of F(ab')2 fragment of antibody 3.2.52
DVVMTQTPLTLSVTIGQPVSISCKSSQSLLYTNGKTFLNWLFQRPGQSPKRLIYMVSKLESGVPDRFSGSGSGTDFTLKISRVEAEDLGIYYCMQSTHFPLTFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 11 - protein, 30 aa, synthetic construct
  REGION 1..30: note = FW(HC)1 of VH of antibody 3.2.52
EVQLQQSGPELVKLGASVRVSCRTSGFTIT

SEQ ID NO: 12 - protein, 14 aa, synthetic construct
  REGION 1..14: note = FW(HC)2 of VH of antibody 3.2.52
WVKQSHGKSLEWIG

SEQ ID NO: 13 - protein, 32 aa, synthetic construct
  REGION 1..32: note = FW(HC)3 of VH of antibody 3.2.52
KATLTGDKSSSTVYMDLRSLTSEGSTVNYCVR

SEQ ID NO: 14 - protein, 11 aa, synthetic construct
  REGION 1..11: note = FW(HC)4 OF VH OF ANTIBODY 3.2.52
WGQGTTLTVSS

SEQ ID NO: 15 - protein, 23 aa, synthetic construct
  REGION 1..23: note = FW(LC)1 of VL of antibody 3.2.52
DVVMTQTPLTLSVTIGQPVSISC

SEQ ID NO: 16 - protein, 15 aa, synthetic construct
  REGION 1..15: note = FW(LC)2 of VL of antibody 3.2.52
WLFQRPGQSPKRLIY

SEQ ID NO: 17 - protein, 32 aa, synthetic construct
  REGION 1..32: note = FW(LC)3 of VL of antibody 3.2.52
GVPDRFSGSGSGTDFTLKISRVEAEDLGIYYC

SEQ ID NO: 18 - protein, 10 aa, synthetic construct
  REGION 1..10: note = SEQ ID NO: 18 FW(LC)4 of VL of antibody 3.2.52
FGGGTKLELK

SEQ ID NO: 19 - protein, 117 aa, synthetic construct
  REGION 1..117: note = VH sequence of antibody 21F12
EVQLQQSGPELLKPGASVKISCKASGFTFTDYTMHWMKQSHGKSLEWIGGINPNSGGTIYNEKFKDKATLTVDKSSRTAYMELRSLTSEDSAVYFCTRGVYDGYFYWGQGTLVTVSA

SEQ ID NO: 20 - protein, 112 aa, synthetic construct
  REGION 1..112: note = SEQ ID NO: 20 VL sequence of antibody 21F12
DVVMTQTPLSLPVSLGDQASISCRSSQSLVYSNGNTFLHWYLQKPGQSPKLLIYKVSTRFSGVPDRFSGSGSGSDFTLKISRVEAEDLGIYFCSQTTHAPFTFGSGTKLAIR

SEQ ID NO: 21 - protein, 119 aa, synthetic construct
  REGION 1..119: note = VH sequence of mouse 3D6
EVKLVESGGGLVKPGASLKLSCAASGFTFSNYGMSWVRQNSDKRLEWVASIRSGGGRTYYSDNVKGRFTISRENAKNTLYLQMSSLKSEDTALYYCVRYDHYSGSSDYWGQGTTVTVSS

SEQ ID NO: 22 - protein, 112 aa, synthetic construct
  REGION 1..112: note = VL sequence of mouse 3D6
YVVMTQTPLTLSVTIGQPASISCKSSQSLLDSDGKTYLNWLLQRPGQSPKRLIYLVSKLDSGVPDRFTGSGSGTDFTLKISRIEAEDLGLYYCWQGTHFPRTFGGGTKLEIK

SEQ ID NO: 23 - protein, 454 aa, synthetic construct
  REGION 1..454: note = Heavy chain sequence of 3.2.52
EVQLQQSGPELVKLGASVRVSCRTSGFTITEYTMHWVKQSHGKSLEWIGGINPITGGSRNNLKFKDKATLTGDKSSSTVYMDLRSLTSEGSTVNYCVRGRTMGPLDYWGQGTTLTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 24 - protein, 8 aa, Homo sapiens
MVGGVVIA